(19) United States
(12) Patent Application Publication
(10) Pub. No.: US 2021/0002657 A1
(43) Pub. Date: Jan. 7, 2021

(54) PLANT HEALTH ASSAY

(71) Applicant: PIONEER HI-BRED INTERNATIONAL, INC., JOHNSTON, IA (US)

(72) Inventors: STEVEN HENRY BASS, HILLSBOROUGH, CA (US); HYEON-JE CHO, ANKENY, IA (US); MYEONG-JE CHO, SUNNYVALE, CA (US); VIRGINIA CRANE, DES MOINES, IA (US); MATTHEW J HECKERT, UNION CITY, CA (US); JIAN JIN, WEST LAFAYETTE, IN (US); TODD J JONES, JOHNSTON, IA (US); KEVIN E MCBRIDE, DAVIS, CA (US); JEANNE SANDAHL, WEST DES MOINES, IA (US); SHIV BAHADUR TIWARI, SAN JOSE, CA (US)

(73) Assignee: PIONEER HI-BRED INTERNATIONAL, INC., JOHNSTON, IA (US)

(21) Appl. No.: 16/977,020

(22) PCT Filed: Feb. 28, 2019
(86) PCT No.: PCT/US19/20079

§ 371 Date: Aug. 31, 2020

Related U.S. Application Data
(60) Provisional application No. 62/637,691, filed on Mar. 2, 2018.

(51) Int. Cl. C12N 15/82; G01N 33/50; C12N 15/81; C12Q 1/06

(52) U.S. Cl. CPC C12N 15/8209; C12Q 1/06; C12N 15/81; G01N 33/5097

(57) ABSTRACT

Methods of detecting the impacts on plant health attributable to the presence of one or more agronomically important polypeptides of interest in a transgenic plant are disclosed. The methods involve transforming plants or plant cells with nucleic acid sequences encoding proteins of agronomically important traits. The transformed plants or plant cells expressing the nucleic acid sequences encoding the proteins of agronomically important traits are compared to transformed plants or plant cells expressing a neutral control gene to detect the impacts on plant health attributable to the presence of the one or more agronomically important polypeptides of interest.

### CROSS-REFERENCE TO RELATED APPLICATIONS

This application claims the benefit of PCT Application Serial Number PCT/US2019/020079, filed Feb. 28, 2019, which claims the benefit of U.S. Provisional Application No. 62/637,691, filed Mar. 2, 2018, both of which are hereby incorporated herein in their entireties by reference.

### FIELD OF THE DISCLOSURE

This disclosure relates to the field of molecular biology. Provided are novel methods of detecting the impact on plant health of recombinant proteins expressed in transgenic plants.

### BACKGROUND

Transformation of a variety of agronomically important plants, e.g., maize, soybean, canola, wheat, Indica rice, sugarcane, sorghum, and inbred lines, with a variety of agronomically important traits, e.g., pest resistance, herbicide resistance, stress tolerance, drought resistance, nitrogen use efficiency (NUE), disease resistance, and those affecting metabolic pathways, continues to be both difficult and time consuming. Some transgenic plants expressing the proteins of these agronomically important traits exhibit undesirable phenotypic responses at different development stages or under different conditions, for example when a protein is expressed at a high level, which may lead to the necessity of abandoning commercial development of an agronomically important trait, oftentimes after considerable resources and manpower have been spent.

Accordingly, there remains a need for new methods of detecting the impact on plant health of recombinant proteins expressed in transgenic plants.

### SUMMARY

In an aspect, the disclosure provides a method of determining an impact on plant health of a gene of interest comprising: a) providing a first plant cell and a second plant cell; b) transforming the first plant cell with a first cassette comprising a gene of interest; c) transforming the second plant cell with a second cassette comprising a neutral control gene; d) culturing i) the first transformed plant cell for expression of the gene of interest; and ii) the second transformed plant cell for expression of the neutral control gene; and e) determining the impact of expression of the gene of interest on plant health relative to expression of the neutral control gene. In a further aspect, the first plant cell and the second plant cell is selected from the group of an alfalfa plant, an Arabidopsis plant, a barley plant, a broad bean plant, a broccoli plant, a bush bean plant, a cabbage plant, a canola plant, a cassava plant, a cauliflower plant, a clover plant, a cotton plant, a kale plant, a maize plant, a millet plant, a mustard plant, an oat plant, a pea plant, a rice plant, a rye plant, a safflower plant, a Setaria plant, a sorghum plant, a soybean plant, a sugarcane plant, a sunflower plant, a switchgrass plant, a tobacco plant, a tomato plant, a triticale plant, a turf grass plant, and a wheat plant. In a further aspect, the first plant cell and the second plant cell is from the same plant. In a further aspect, the first plant cell and the second plant cell of the maize plant is an immature embryo. In a further aspect, the first plant cell and the second plant cell of the bush bean plant is a leaf. In a further aspect, the first plant cell and the second plant cell of the soybean plant is a leaf. In a further aspect, the first plant cell and the second plant cell of the soybean plant is an immature cotyledon. In a further aspect, the first plant cell and the second plant cell of the soybean plant is an imbibed mature cotyledon. In a further aspect, the first plant cell and the second plant cell of the soybean plant is an embryonic axis. In a further aspect, the gene of interest is selected from the group of a gene conferring pest resistance, herbicide resistance, stress tolerance, drought resistance, nitrogen use efficiency (NUE), disease resistance, and an ability to alter a metabolic pathway. In a further aspect, the neutral control gene is selected from the group of a chloramphenicol acetyl transferase (CAT) gene, a fluorescent protein (FP) gene, a phosphomannose isomerase (PMI) gene, a β-glucuronidase (GUS) gene, and a housekeeping gene. In a further aspect, the first cassette further comprises a promoter operably linked to the gene of interest for expression of the gene of interest in the first plant cell and the second cassette further comprises a promoter operably linked to the neutral control gene for expression of the neutral control gene in the second plant cell. In a further aspect, the promoter of the first cassette and the promoter of the second cassette is the same promoter. In a further aspect, determining the impact of expression of the gene of interest on plant health relative to expression of the neutral control gene is a visual observation of a plant tissue. In a further aspect, the visual observation is selected from the group of anthocyanin pigment production of the plant tissue, browning of the plant tissue, necrosis of the plant tissue, and growth of the plant tissue. In a further aspect, wherein the first plant cell and the second plant cell of the maize plant is an immature embryo, the gene of interest is selected from the group of a gene conferring pest resistance, herbicide resistance, stress tolerance, drought resistance, nitrogen use efficiency (NUE), disease resistance, and an ability to alter a metabolic pathway. In a further aspect, the neutral control gene is selected from the group of a chloramphenicol acetyl transferase (CAT) gene, a fluorescent protein (FP) gene, a phosphomannose isomerase (PMI) gene, a β-glucuronidase (GUS) gene, and a housekeeping gene. In a further aspect, the first cassette further comprises a promoter operably linked to the gene of interest for expression of the gene of interest in the first plant cell and the second cassette further comprises a promoter operably linked to the neutral control gene for expression of the neutral control gene in the second plant cell. In a further aspect, the promoter of the first cassette and the promoter of the second cassette is the same promoter. In a further aspect, determining the impact of expression of the gene of interest on plant health relative to expression of the neutral control gene is a visual observation of a plant tissue. In a further aspect, the visual observation is selected from the group of anthocyanin pigment production of the plant tissue, browning of the plant tissue, necrosis of the plant tissue, and growth of the plant tissue.

In an aspect, the present disclosure provides a method of determining the impact on plant health of a gene of interest comprising: a) providing a first plant cell and a second plant cell; b) transforming the first plant cell with a first cassette comprising a gene of interest and a third cassette comprising a reporter gene; c) transforming the second plant cell with a second cassette comprising a neutral control gene and the third cassette comprising the reporter gene; d) culturing i) the first transformed plant cell for expression of the reporter gene and the of the gene of interest; and ii) the second transformed plant cell for expression of the reporter gene and the neutral control gene; and e) determining the impact of expression of the gene of interest on plant health by measuring expression of the reporter gene and the gene of interest relative to expression of the reporter gene and the neutral control gene. In a further aspect, the first plant cell and the second plant cell is from the same plant. In a further aspect, the same plant is a monocot plant or a dicot plant. In a further aspect, the monocot plant is selected from the group of a barley plant, a maize plant, a millet plant, an oat plant, a rice plant, a rye plant, a Setaria plant, a sorghum plant, a sugarcane plant, a switchgrass plant, a triticale plant, a turf grass plant, and a wheat plant. In a further aspect, the dicot plant is selected from the group of an alfalfa plant, an Arabidopsis plant, a broad bean plant, a broccoli plant, a bush bean plant, a cabbage plant, a canola plant, a cassava plant, a cauliflower plant, a clover plant, a cotton plant, a kale plant, a mustard plant, an oat plant, a pea plant, a rice plant, a rye plant, a safflower plant, a soybean plant, a sunflower plant, a tobacco plant, and a tomato plant. In a further aspect, the first plant cell and the second plant cell is selected from the group of a maize leaf, a maize immature embryo, a maize immature zygotic embryo, a bush bean leaf, a soybean leaf, a soybean immature cotyledon, a soybean imbibed mature cotyledon, a soybean embryonic axis, a tobacco leaf, an Arabidopsis leaf, and a Setaria leaf In a further aspect, the first plant cell and the second plant cell is a protoplast derived from an Arabidopsis leaf or a maize leaf. In a further aspect, the first plant cell and the second plant cell is the maize immature embryo. In a further aspect, the first plant cell and the second plant cell is the bush bean leaf In a further aspect, the first plant cell and the second plant cell is the soybean leaf. In a further aspect, the first plant cell and the second plant cell is the soybean immature cotyledon. In a further aspect, the first plant cell and the second plant cell is the soybean imbibed mature cotyledon. In a further aspect, the first plant cell and the second plant cell is the soybean embryonic axis. In a further aspect, the gene of interest is selected from the group of a gene conferring pest resistance, herbicide resistance, stress tolerance, drought resistance, nitrogen use efficiency (NUE), disease resistance, and an ability to alter a metabolic pathway. In a further aspect, the neutral control gene is selected from the group of a chloramphenicol acetyl transferase (CAT) gene, a fluorescent protein (FP) gene, a phosphomannose isomerase (PMI) gene, a β-glucuronidase (GUS) gene, and a housekeeping gene. In a further aspect, the reporter gene is selected from the group of an ATP dependent luciferase gene, an ATP independent luciferase, a chloramphenicol acetyl transferase (CAT) gene, a fluorescent protein (FP) gene, a β-glucuronidase (GUS) gene, a β-galactosidase (GAL) gene, and an alkaline phosphatase gene. In a further aspect, the first cassette, the second cassette and the third cassette further comprises a promoter. In a further aspect, the promoter of the first cassette and the promoter of the second cassette is the same promoter, and the promoter of the third cassette is the same as or different from the promoter of the first and second cassette. In a further aspect, the promoter of the third cassette is different from the promoter of the first and second cassette. In a further aspect, the first cassette is on a first vector, the second cassette is on a second vector, and the third cassette is on a third vector. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health. In a further aspect, the first cassette and the second cassette further comprises a promoter. In a further aspect, the promoter of the first cassette and the promoter of the second cassette is the same promoter. In a further aspect, the first cassette and the third cassette is on a first vector and the second cassette and the third cassette is on a second vector. In a further aspect, the reporter gene of the third cassette and the gene of interest of the first cassette are expressed as a translational fusion protein and the reporter gene of the third cassette and the neutral control gene of the second cassette are expressed as a translational fusion protein. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health. In a further aspect, wherein the first plant cell and the second plant cell is a protoplast derived from an Arabidopsis leaf or a maize leaf, the gene of interest is selected from the group of a gene conferring pest resistance, herbicide resistance, stress tolerance, drought resistance, nitrogen use efficiency (NUE), disease resistance, and an ability to alter a metabolic pathway. In a further aspect, the neutral control gene is selected from the group of a chloramphenicol acetyl transferase (CAT) gene, a fluorescent protein (FP) gene, a phosphomannose isomerase (PMI) gene, a β-glucuronidase (GUS) gene, and a housekeeping gene. In a further aspect, the reporter gene is selected from the group of an ATP dependent luciferase gene, an ATP independent luciferase, a chloramphenicol acetyl transferase (CAT) gene, a fluorescent protein (FP) gene, a β-glucuronidase (GUS) gene, a β-galactosidase (GAL) gene, and an alkaline phosphatase gene. In a further aspect, the first cassette, the second cassette and the third cassette further comprises a promoter. In a further aspect, the promoter of the first cassette and the promoter of the second cassette is the same promoter, and the promoter of the third cassette is the same as or different from the promoter of the first and second cassette. In a further aspect, the promoter of the third cassette is different from the promoter of the first and second cassette. In a further aspect, the first cassette is on a first vector, the second cassette is on a second vector, and the third cassette is on a third vector. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health. In a further aspect, the first cassette and the second cassette further comprises a promoter. In a further aspect, the promoter of the first cassette and the promoter of the second cassette is the same promoter. In a further aspect, the first cassette and the third cassette is on a first vector and the second cassette and the third cassette is on a second vector. In a further aspect, the reporter gene of the third cassette and the gene of interest of the first cassette are expressed as a translational fusion protein and the reporter gene of the third cassette and the neutral control gene of the second cassette are expressed as a translational fusion protein. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health.

In an aspect, the disclosure provides a method of determining the impact on plant health of a gene of interest comprising: a) providing a first plant cell and a second plant cell; b) transforming the first plant cell with a first cassette comprising a gene of interest, a third cassette comprising a reporter gene, and a fourth cassette comprising a morphogenic gene; c) transforming the second plant cell with a second cassette comprising a neutral control gene, the third cassette comprising the reporter gene, and the fourth cassette comprising the morphogenic gene; d) culturing i) the first transformed plant cell for expression of the reporter gene and the gene of interest; and ii) the second transformed plant cell for expression of the reporter gene and the neutral control gene; and e) determining the impact of expression of the gene of interest on plant health by measuring expression of the reporter gene and the gene of interest relative to expression of the reporter gene and the neutral control gene. In a further aspect, the first plant cell and the second plant cell is from the same plant. In a further aspect, the same plant is a monocot plant or a dicot plant. In a further aspect, the monocot plant is selected from the group of a barley plant, a maize plant, a millet plant, an oat plant, a rice plant, a rye plant, a Setaria plant, a sorghum plant, a sugarcane plant, a switchgrass plant, a triticale plant, a turf grass plant, and a wheat plant. In a further aspect, the dicot plant is selected from the group of an alfalfa plant, an Arabidopsis plant, a broad bean plant, a broccoli plant, a bush bean plant, a cabbage plant, a canola plant, a cassava plant, a cauliflower plant, a clover plant, a cotton plant, a kale plant, a mustard plant, an oat plant, a pea plant, a rice plant, a rye plant, a safflower plant, a soybean plant, a sunflower plant, a tobacco plant, and a tomato plant. In a further aspect, the first plant cell and the second plant cell is selected from the group of a maize leaf, a maize immature embryo, a bush bean leaf, a soybean leaf, a soybean immature cotyledon, a soybean imbibed mature cotyledon, a soybean embryonic axis, a tobacco leaf, an Arabidopsis leaf, and a Setaria leaf. In a further aspect, the first plant cell and the second plant cell is a protoplast derived from an Arabidopsis leaf or a maize leaf In a further aspect, the first plant cell and the second plant cell is the maize immature embryo. In a further aspect, the first plant cell and the second plant cell is the bush bean leaf. In a further aspect, the first plant cell and the second plant cell is the soybean leaf. In a further aspect, the first plant cell and the second plant cell is the soybean immature cotyledon. In a further aspect, the first plant cell and the second plant cell is the soybean imbibed mature cotyledon. In a further aspect, the first plant cell and the second plant cell is the soybean embryonic axis. In a further aspect, the gene of interest is selected from the group of a gene conferring pest resistance, herbicide resistance, stress tolerance, drought resistance, nitrogen use efficiency (NUE), disease resistance, and an ability to alter a metabolic pathway. In a further aspect, the neutral control gene is selected from the group of a chloramphenicol acetyl transferase (CAT) gene, a fluorescent protein (FP) gene, a phosphomannose isomerase (PMI) gene, a β-glucuronidase (GUS) gene, and a housekeeping gene. In a further aspect, the reporter gene is selected from the group of an ATP dependent luciferase gene, a chloramphenicol acetyl transferase (CAT) gene, a fluorescent protein (FP) gene, a β-glucuronidase (GUS) gene, a β-galactosidase (GAL) gene, and an alkaline phosphatase gene. In a further aspect, the morphogenic gene is selected from the group of a WUS1 gene, a WUS2 gene, a WUS3 gene, a WOX2A gene, a WOX4 gene, a WOX5 gene, a WOX9 gene, a MYB118 gene, a MYB115 gene, a BABYBOOM gene, a CLAVATA gene, a LEC1 gene, a LEC2 gene, a KN1/STM gene, an IPT gene, a MONOPTEROS-DELTA gene, an Agrobacterium AV-6b gene, an Agrobacterium IAA-h gene, an Agrobacterium IAA-m gene, an Arabidopsis SERK gene, and an Arabidopsis AGL15 gene. In a further aspect, the first cassette, the second cassette, the third cassette, and the fourth cassette further comprises a promoter. In a further aspect, the promoter of the first cassette and the promoter of the second cassette is the same promoter, the promoter of the third cassette is the same as or different from the promoter of the first and second cassette and the fourth cassette, and the promoter of the fourth cassette is the same as or different from the promoter of the first and second cassette and the third cassette. In a further aspect, the promoter of the third cassette is different from the promoter of the first and second cassette and the fourth cassette, and the promoter of the fourth cassette is different from the promoter of the first and second cassette and the third cassette. In a further aspect, the first cassette is on a first vector, the second cassette is on a second vector, the third cassette is on a third vector, and the fourth cassette is on a fourth vector. In a further aspect, the reporter gene is an ATP dependent luciferase gene. In a further aspect, wherein the reporter gene is an ATP dependent luciferase gene the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health. In a further aspect, the first cassette and the second cassette further comprises a promoter. In a further aspect, the promoter of the first cassette and the promoter of the second cassette is the same promoter. In a further aspect, the first cassette, the third cassette, and the fourth cassette is on a first vector and the second cassette, the third cassette, and the fourth cassette is on a second vector. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health.

In an aspect, the disclosure provides a method of determining the impact on plant health of a gene of interest comprising: a) providing a first plant cell and a second plant cell; b) transforming the first plant cell with a first cassette comprising a gene of interest, a third cassette comprising a reporter gene, and a fourth cassette comprising a morphogenic gene; c) transforming the second plant cell with a second cassette comprising a neutral control gene, the third cassette comprising the reporter gene, and the fourth cassette comprising the morphogenic gene; d) culturing i) the first transformed plant cell for expression of the reporter gene and the gene of interest; and ii) the second transformed plant cell for expression of the reporter gene and the neutral control gene; and e) determining the impact of expression of the gene of interest on plant health by measuring expression of the reporter gene and the gene of interest relative to expression of the reporter gene and the neutral control gene. In a further aspect, the first plant cell and the second plant cell is from the same plant. In a further aspect, the same plant is a monocot plant or a dicot plant. In a further aspect, the monocot plant is selected from the group of a barley plant, a maize plant, a millet plant, an oat plant, a rice plant, a rye plant, a Setaria plant, a sorghum plant, a sugarcane plant, a switchgrass plant, a triticale plant, a turf grass plant, and a wheat plant. In a further aspect, the dicot plant is selected from the group of an alfalfa plant, an Arabidopsis plant, a broad bean plant, a broccoli plant, a bush bean plant, a cabbage plant, a canola plant, a cassava plant, a cauliflower plant, a clover plant, a cotton plant, a kale plant, a mustard plant, an oat plant, a pea plant, a rice plant, a rye plant, a safflower plant, a soybean plant, a sunflower plant, a tobacco plant, and a tomato plant. In a further aspect, the first plant cell and the second plant cell is selected from the group of a maize leaf, a maize immature embryo, a bush bean leaf, a soybean leaf, a soybean immature cotyledon, a soybean imbibed mature cotyledon, a soybean embryonic axis, a tobacco leaf, an Arabidopsis leaf, and a Setaria leaf. In a further aspect, the first plant cell and the second plant cell is a protoplast derived from an Arabidopsis leaf or a maize leaf In a further aspect, the first plant cell and the second plant cell is the maize immature embryo. In a further aspect, the first plant cell and the second plant cell is the bush bean leaf. In a further aspect, the first plant cell and the second plant cell is the soybean leaf. In a further aspect, the first plant cell and the second plant cell is the soybean immature cotyledon. In a further aspect, the first plant cell and the second plant cell is the soybean imbibed mature cotyledon. In a further aspect, the first plant cell and the second plant cell is the soybean embryonic axis. In a further aspect, the gene of interest is selected from the group of a gene conferring pest resistance, herbicide resistance, stress tolerance, drought resistance, nitrogen use efficiency (NUE), disease resistance, and an ability to alter a metabolic pathway. In a further aspect, the neutral control gene is selected from the group of a chloramphenicol acetyl transferase (CAT) gene, a fluorescent protein (FP) gene, a phosphomannose isomerase (PMI) gene, a β-glucuronidase (GUS) gene, and a housekeeping gene. In a further aspect, the reporter gene is selected from the group of an ATP dependent luciferase gene, a chloramphenicol acetyl transferase (CAT) gene, a fluorescent protein (FP) gene, a β-glucuronidase (GUS) gene, a β-galactosidase (GAL) gene, and an alkaline phosphatase gene. In a further aspect. the morphogenic gene is selected from the group of a WUS1 gene, a WUS2 gene, a WUS3 gene, a WOX2A gene, a WOX4 gene, a WOX5 gene, a WOX9 gene, a MYB118 gene, a MYB115 gene, a BABYBOOM gene, a CLAVATA gene, a LEC1 gene, a LEC2 gene, a KN1/STM gene, an IPT gene, a MONOPTEROS-DELTA gene, an Agrobacterium AV-6b gene, an Agrobacterium IAA-h gene, an Agrobacterium IAA-m gene, an Arabidopsis SERK gene, and an Arabidopsis AGL15 gene. In a further aspect, the first cassette, the second cassette, the third cassette, and the fourth cassette further comprises a promoter. In a further aspect, the promoter of the first cassette and the promoter of the second cassette is the same promoter, the promoter of the third cassette is the same as or different from the promoter of the first and second cassette and the fourth cassette, and the promoter of the fourth cassette is the same as or different from the promoter of the first and second cassette and the third cassette. In a further aspect, the promoter of the third cassette is different from the promoter of the first and second cassette and the fourth cassette, and the promoter of the fourth cassette is different from the promoter of the first and second cassette and the third cassette. In a further aspect, the first cassette is on a first vector, the second cassette is on a second vector, the third cassette is on a third vector, and the fourth cassette is on a fourth vector. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health. In a further aspect, the first cassette and the second cassette further comprises a promoter. In a further aspect, the promoter of the first cassette and the promoter of the second cassette is the same promoter. In a further aspect, the first cassette, the third cassette, and the fourth cassette is on a first vector and the second cassette, the third cassette, and the fourth cassette is on a second vector. In a further aspect, the reporter gene of the third cassette and the gene of interest of the first cassette are expressed as a translational fusion protein and the reporter gene of the third cassette and the neutral control gene of the second cassette are expressed as a translational fusion protein. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health. In a further aspect, wherein the first plant cell and the second plant cell is a protoplast derived from an Arabidopsis leaf or a maize leaf, the gene of interest is selected from the group of a gene conferring pest resistance, herbicide resistance, stress tolerance, drought resistance, nitrogen use efficiency (NUE), disease resistance, and an ability to alter a metabolic pathway. In a further aspect, the neutral control gene is selected from the group of a chloramphenicol acetyl transferase (CAT) gene, a fluorescent protein (FP) gene, a phosphomannose isomerase (PMI) gene, a β-glucuronidase (GUS) gene, and a housekeeping gene. In a further aspect, the reporter gene is selected from the group of an ATP dependent luciferase gene, a chloramphenicol acetyl transferase (CAT) gene, a fluorescent protein (FP) gene, a β-glucuronidase (GUS) gene, a β-galactosidase (GAL) gene, and an alkaline phosphatase gene. In a further aspect. the morphogenic gene is selected from the group of a WUS1 gene, a WUS2 gene, a WUS3 gene, a WOX2A gene, a WOX4 gene, a WOX5 gene, a WOX9 gene, a MYB118 gene, a MYB115 gene, a BABYBOOM gene, a CLAVATA gene, a LEC1 gene, a LEC2 gene, a KN1/STM gene, an IPT gene, a MONOPTEROS-DELTA gene, an Agrobacterium AV-6b gene, an Agrobacterium IAA-h gene, an Agrobacterium IAA-m gene, an Arabidopsis SERK gene, and an Arabidopsis AGL15 gene. In a further aspect, the first cassette, the second cassette, the third cassette, and the fourth cassette further comprises a promoter. In a further aspect, the promoter of the first cassette and the promoter of the second cassette is the same promoter, the promoter of the third cassette is the same as or different from the promoter of the first and second cassette and the fourth cassette, and the promoter of the fourth cassette is the same as or different from the promoter of the first and second cassette and the third cassette. In a further aspect, the promoter of the third cassette is different from the promoter of the first and second cassette and the fourth cassette, and the promoter of the fourth cassette is different from the promoter of the first and second cassette and the third cassette. In a further aspect, the first cassette is on a first vector, the second cassette is on a second vector, the third cassette is on a third vector, and the fourth cassette is on a fourth vector. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health. In a further aspect, the first cassette and the second cassette further comprises a promoter. In a further aspect, the promoter of the first cassette and the promoter of the second cassette is the same promoter. In a further aspect, the first cassette, the third cassette, and the fourth cassette is on a first vector and the second cassette, the third cassette, and the fourth cassette is on a second vector. In a further aspect, the reporter gene of the third cassette and the gene of interest of the first cassette are expressed as a translational fusion protein and the reporter gene of the third cassette and the neutral control gene of the second cassette are expressed as a translational fusion protein. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health. In a further aspect, wherein the first plant cell and the second plant cell is the maize immature embryo, the gene of interest is selected from the group of a gene conferring pest resistance, herbicide resistance, stress tolerance, drought resistance, nitrogen use efficiency (NUE), disease resistance, and an ability to alter a metabolic pathway. In a further aspect, the neutral control gene is selected from the group of a chloramphenicol acetyl transferase (CAT) gene, a fluorescent protein (FP) gene, a phosphomannose isomerase (PMI) gene, a β-glucuronidase (GUS) gene, and a housekeeping gene. In a further aspect, the reporter gene is selected from the group of an ATP dependent luciferase gene, a chloramphenicol acetyl transferase (CAT) gene, a fluorescent protein (FP) gene, a β-glucuronidase (GUS) gene, a β-galactosidase (GAL) gene, and an alkaline phosphatase gene. In a further aspect. the morphogenic gene is selected from the group of a WUS1 gene, a WUS2 gene, a WUS3 gene, a WOX2A gene, a WOX4 gene, a WOX5 gene, a WOX9 gene, a MYB118 gene, a MYB115 gene, a BABYBOOM gene, a CLAVATA gene, a LEC1 gene, a LEC2 gene, a KN1/STM gene, an IPT gene, a MONOPTEROS-DELTA gene, an Agrobacterium AV-6b gene, an Agrobacterium IAA-h gene, an Agrobacterium IAA-m gene, an Arabidopsis SERK gene, and an Arabidopsis AGL15 gene. In a further aspect, the first cassette, the second cassette, the third cassette, and the fourth cassette further comprises a promoter. In a further aspect, the promoter of the first cassette and the promoter of the second cassette is the same promoter, the promoter of the third cassette is the same as or different from the promoter of the first and second cassette and the fourth cassette, and the promoter of the fourth cassette is the same as or different from the promoter of the first and second cassette and the third cassette. In a further aspect, the promoter of the third cassette is different from the promoter of the first and second cassette and the fourth cassette, and the promoter of the fourth cassette is different from the promoter of the first and second cassette and the third cassette. In a further aspect, the first cassette is on a first vector, the second cassette is on a second vector, the third cassette is on a third vector, and the fourth cassette is on a fourth vector. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health. In a further aspect, the first cassette and the second cassette further comprises a promoter. In a further aspect, the promoter of the first cassette and the promoter of the second cassette is the same promoter. In a further aspect, the first cassette, the third cassette, and the fourth cassette is on a first vector and the second cassette, the third cassette, and the fourth cassette is on a second vector. In a further aspect, the reporter gene of the third cassette and the gene of interest of the first cassette are expressed as a translational fusion protein and the reporter gene of the third cassette and the neutral control gene of the second cassette are expressed as a translational fusion protein. In a further aspect, the reporter gene is an ATP dependent luciferase gene. In a further aspect, wherein the reporter gene is an ATP dependent luciferase gene, the ATP dependent luciferase gene is expressed and said expression is detected in an assay for ATP dependent luciferase activity performed in the absence of exogenous ATP. In a further aspect, a ratio of the ATP dependent luciferase activity of the first plant cell expressing the gene of interest and the ATP dependent luciferase activity of the second plant cell expressing the neutral control gene indicates plant health. In a further aspect, the ratio below 70% of neutral indicates negative plant cell health. In a further aspect, the reporter gene is a fluorescent protein (FP) gene. In a further aspect, wherein the reporter gene is a fluorescent protein (FP) gene, the fluorescent protein gene is a green or yellow fluorescent protein gene. In a further aspect, a ratio of the green or yellow fluorescent protein gene expression of the first plant cell expressing the gene of interest and the green or yellow fluorescent protein gene expression of the second plant cell expressing the neutral control gene indicates plant health. In a further aspect, the ratio below 70% of neutral indicates negative plant cell health.

### BRIEF DESCRIPTION OF THE FIGURES

FIG. 1 shows a representative vector design for plant response measurement comprising from left to right: a right border (RB); a test gene cassette comprising a gene of interest or a neutral control gene; a selectable marker and reporter gene cassette; and a morphogenic gene cassette.

FIG. 2A shows the vector design used in Example 2.

FIG. 2B shows the growth response of Gene E* 14 d.p.i.

FIG. 2C shows the growth response of Gene E 14 d.p.i.

FIG. 3 shows plant tissue development 4 weeks post infection with Gene E* and Gene E.

FIG. 4A shows the vector design used in Example 3.

FIG. 4B shows plant response in Bush Bean leaves 3 and 6-days post infiltration for Untreated, Empty AGL1; Empty Vector, DMMV driving DsRED2, DMMV driving Gene A, DMMV driving Gene F, DMMV driving Gene G, DMMV driving Gene H, and DMMV driving Gene I leaves.

FIG. 5A shows the vector design used in Example 4.

FIG. 5B-5K shows colony size on glucose vs. galactose for each Test Gene tested: Gene A (FIG. 5B and FIG. 5C); Gene J (FIG. 5D and FIG. 5E); Gene C (FIG. 5F and FIG. 5G); Gene E (FIG. 5H and FIG. 5I); and Gene K (FIG. 5J and FIG. 5K).

FIG. 6 shows that Gene L (squares) had approximately a 20-fold less impact on plant health than Gene A (diamonds).

### DETAILED DESCRIPTION

It is to be understood that this disclosure is not limited to the particular methodology, protocols, cell lines, genera, and reagents described, as such may vary. It is also to be understood that the terminology used herein is for the purpose of describing particular aspects only, and is not intended to limit the scope of the present disclosure.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof, and so forth. All technical and scientific terms used herein have the same meaning as commonly understood to one of ordinary skill in the art to which this disclosure belongs unless clearly indicated otherwise.

The methods of the disclosure detect impacts on plant health and can be used for gene/promoter screening, vector construction, construct optimization, and event selection. The methods of the disclosure to determine impacts on plant health include transient transformation including, transformation of plant derived protoplasts and Agrobacterium infiltration of plant leaf tissue, stable transformation including, rapid transformation, and high throughput plant surrogate yeast transformation.

The present disclosure is drawn to methods of detecting the impact on plant health attributable to the presence of one or more agronomically important genes of interest in a transgenic plant. Non-limiting impacts on plant health include decreased expression of one or more transgenes of interest, decreased plant transformation efficiency and/or low transgene event recovery, decreased crop yield, and negative impacts on plant health, up to, and possibly including, plant death. The methods involve transforming plants with nucleic acid sequences encoding proteins of agronomically important traits. The transformed plants expressing the nucleic acid sequences encoding the proteins of agronomically important traits are compared to transformed plants expressing a neutral control gene to detect impacts on plant health attributable to the presence of the one or more agronomically important polypeptide of interest. Detecting these impacts on plant health allows for more efficient production of agronomically important transgenic plants. The present method also provides a means for rapidly testing gene variants to determine which variants ameliorate the impacts on plant health.

In an embodiment, a method is provided for determining an impact on plant health, including an adverse phenotypic effect attributable to the expression of one or more agronomically important polypeptide of interest in a transgenic plant. The impact on plant health of a gene of interest is determined by providing a first plant cell and a second cell, the first plant cell being transformed with a gene of interest while the second plant cell is transformed with a neutral control gene. The plant cells are cultured to permit expression of the gene of interest in the first transformed plant cell and expression of the neutral control gene in the second transformed plant cell. The impact on plant health of the gene of interest is determined by the comparison of the expression of the neutral control gene to the expression of the gene of interest. This comparison may be determined by a visual observation of the transformed plant cells. Visual observations include, but are not limited to, anthocyanin pigment production of the plant tissue, browning of the plant tissue, necrosis of the plant tissue, and growth of the plant tissue.

The present disclosure provides novel methods for detecting impacts on plant health attributable to the expression of one or more agronomically important polypeptide of interest in a transgenic plant or a transgenic plant cell. The term “plant” refers to whole plants, plant organs (e.g., leaves, stems, roots, etc.), plant tissues, plant cells, plant parts, seeds, propagules, embryos and progeny of the same. Plant cells can be differentiated or undifferentiated (e.g. callus, undifferentiated callus, immature and mature embryos, immature zygotic embryo, immature and mature cotyledon, embryonic axis, suspension culture cells, protoplasts, leaf, leaf cells, root cells, phloem cells and pollen). Plant cells include, without limitation, cells from seeds, suspension cultures, explants, immature embryos, embryos, zygotic embryos, somatic embryos, embryogenic callus, meristem, somatic meristems, organogenic callus, protoplasts, embryos derived from mature ear-derived seed, leaf bases, leaves from mature plants, leaf tips, immature influorescences, tassel, immature ear, silks, cotyledons, immature and mature cotyledons, embryonic axes, meristematic regions, callus tissue, cells from leaves, cells from stems, cells from roots, cells from shoots, gametophytes, sporophytes, pollen and microspores. Plant parts include differentiated and undifferentiated tissues including, but not limited to, roots, stems, shoots, leaves, pollen, seeds, tumor tissue and various forms of cells in culture (e. g., single cells, protoplasts, embryos, and callus tissue). The plant tissue may be in a plant or in a plant organ, tissue, or cell culture.

The plant cells used in the disclosed methods can be derived from a monocot plant, including, but not limited to, barley, maize (corn), millet (e.g., pearl millet (Pennisetum glaucum), proso millet (Panicum miliaceum), foxtail millet (Setaria italica), finger millet (Eleusine coracana)), oats, rice, rye, Setaria sp., sorghum, triticale, or wheat, or leaf and stem crops, including, but not limited to, bamboo, marram grass, meadow-grass, reeds, ryegrass, sugarcane; lawn grasses, ornamental grasses, and other grasses such as switchgrass and turf grass. Alternatively, the plant cells used in the disclosed methods can be derived from a dicot plant, including, but not limited to, kale, cauliflower, broccoli, mustard plant, cabbage, pea, clover, alfalfa, broad bean, tomato, peanut, cassava, soybean, canola, alfalfa, sunflower, safflower, tobacco, Arabidopsis, or cotton.

The cells of any plant, including higher plants, e.g., classes of Angiospermae and Gymnospermae may be used in the methods of the disclosure. Plant cells of the subclasses of the Dicotylodenae and the Monocotyledonae are suitable for use in the methods of the disclosure. Plant cells of suitable species useful in the methods of the disclosure may come from the family Acanthaceae, Alliaceae, Alstroemeriaceae, Amaryllidaceae, Apocynaceae, Arecaceae, Asteraceae, Berberidaceae, Bixaceae, Brassicaceae, Bromeliaceae, Cannabaceae, Caryophyllaceae, Cephalotaxaceae, Chenopodiaceae, Colchicaceae, Cucurbitaceae, Dioscoreaceae, Ephedraceae, Erythroxylaceae, Euphorbiaceae, Fabaceae, Lamiaceae, Linaceae, Lycopodiaceae, Malvaceae, Melanthiaceae, Musaceae, Myrtaceae, Nyssaceae, Papaveraceae, Pinaceae, Plantaginaceae, Poaceae, Rosaceae, Rubiaceae, Salicaceae, Sapindaceae, Solanaceae, Taxaceae, Theaceae, and Vitaceae. Plant cells from members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea may be used in the methods of the disclosure.

Plant cells important or interesting for agriculture, horticulture, biomass production (for production of liquid fuel molecules and other chemicals), and/or forestry may be used in the methods of the disclosure. Non-limiting examples include, for instance, Panicum virgatum (switchgrass), Miscanthus giganteus (miscanthus), Saccharum spp. (sugarcane, energycane), Populus balsamifera (poplar), cotton (Gossypium barbadense, Gossypium hirsutum), Helianthus annuus (sunflower), Medicago sativa (alfalfa), Beta vulgaris (sugarbeet), sorghum (Sorghum bicolor, Sorghum vulgare), Erianthus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Salix spp. (willow), Eucalyptus spp. (eucalyptus, including E. grandis (and its hybrids, known as “urograndis”), E. globulus, E. camaldulensis, E. tereticornis, E. viminalis, E. nitens, E. saligna and E. urophylla), Triticosecale spp. (triticum—wheat X rye), Bamboo, Carthamus tinctorius (safflower), Jatropha curcas (jatropha), Ricinus communis (castor), Elaeis guineensis (palm), Linum usitatissimum (flax), Manihot esculenta (cassava), Lycopersicon esculentum (tomato), Lactuca sativa (lettuce), Phaseolus vulgaris (green beans), Phaseolus limensis (lima beans), Lathyrus spp. (peas), Musa paradisiaca (banana), Solanum tuberosum (potato), Brassica spp. (B. napus (canola), B. rapa, B. juncea), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Arachis hypogaea (peanuts), Ipomoea batatus (sweet potato), Cocos nucifera (coconut), Citrus spp. (citrus trees), Persea americana (avocado), fig (Ficus casica), guava (Psidium guajava), mango (Mangifera Indica), olive (Olea europaea), Carica papaya (papaya), Anacardium occidentale (cashew), Macadamia integrifolia (macadamia tree), Prunus amygdalus (almond), Allium cepa (onion), Cucumis melo (musk melon), Cucumis sativus (cucumber), Cucumis cantalupensis (cantaloupe), Cucurbita maxima (squash), Cucurbita moschata (squash), Spinacea oleracea (spinach), Citrullus lanatus (watermelon), Abelmoschus esculentus (okra), Solanum melongena (eggplant), Cyamopsis tetragonoloba (guar bean), Ceratonia siliqua (locust bean), Trigonella foenum-graecum (fenugreek), Vigna radiata (mung bean), Vigna unguiculata (cowpea), Vicia faba (fava bean), Cicer arietinum (chickpea), Lens culinaris (lentil), Papaver somniferum (opium poppy), Papaver orientale, Taxus baccata, Taxus brevifolia, Artemisia annua, Cannabis sativa, Camptotheca acuminate, Catharanthus roseus, Vinca rosea, Cinchona officinalis, Colchicum autumnale, Veratrum californica., Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana (achiote), Alstroemeria spp., Rosa spp. (rose), Rhododendron spp. (azalea), Macrophylla hydrangea (hydrangea), Hibiscus rosasanensis (hibiscus), Tulipa spp. (tulips), Narcissus spp. (daffodils), Petunia hybrida (petunias), Dianthus caryophyllus (carnation), Euphorbia pulcherrima (poinsettia), chrysanthemum, Nicotiana tabacum (tobacco), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass), Phleum pratense (timothy), and conifers.

Conifers may be used in the methods of the disclosure and include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Eastern or Canadian hemlock (Tsuga canadensis); Western hemlock (Tsuga heterophylla); Mountain hemlock (Tsuga mertensiana); Tamarack or Larch (Larix occidentalis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Turf grasses may be used in the methods of the disclosure and include, but are not limited to: annual bluegrass (Poa annua); annual ryegrass (Lolium multiflorum); Canada bluegrass (Poa compressa); colonial bentgrass (Agrostis tenuis); creeping bentgrass (Agrostis palustris); crested wheatgrass (Agropyron desertorum); fairway wheatgrass (Agropyron cristatum); hard fescue (Festuca longifolia); Kentucky bluegrass (Poa pratensis); orchardgrass (Dactylis glomerata); perennial ryegrass (Lolium perenne); red fescue (Festuca rubra); redtop (Agrostis alba); rough bluegrass (Poa trivialis); sheep fescue (Festuca ovina); smooth bromegrass (Bromus inermis); timothy (Phleum pratense); velvet bentgrass (Agrostis canina); weeping alkaligrass (Puccinellia distans); western wheatgrass (Agropyron smithii); St. Augustine grass (Stenotaphrum secundatum); zoysia grass (Zoysia spp.); Bahia grass (Paspalum notatum); carpet grass (Axonopus affinis); centipede grass (Eremochloa ophiuroides); kikuyu grass (Pennisetum clandesinum); seashore paspalum (Paspalum vaginatum); blue gramma (Bouteloua gracilis); buffalo grass (Buchloe dactyloids); sideoats gramma (Bouteloua curtipendula).

A transgenic plant is defined as a mature, fertile plant that contains a transgene.

The methods of the disclosure involve introducing a polypeptide or polynucleotide of interest into a plant or plant cell for testing to detect the impacts on plant health attributable to the presence of one or more agronomically important genes of interest in the transgenic plant or transgenic plant cell. “Introducing” is as used herein means presenting to the plant or plant cell the polynucleotide or polypeptide in such a manner that the sequence gains access to the interior of the plant or a cell of the plant. The methods of the disclosure do not depend on a particular method for introducing a polynucleotide or polypeptide into a plant, only that the polynucleotide(s) or polypeptide(s) gains access to the interior of at least one cell of the plant. Methods for introducing polynucleotide(s) or polypeptide(s) into plants are known in the art including, but not limited to, stable transformation methods, transient transformation methods, and virus-mediated methods.

“Stable transformation” as used herein means that a cassette containing a polynucleotide of interest introduced into a plant or a plant cell integrates into the genome of the plant or the plant cell and is capable of being inherited by the progeny thereof “Transient transformation” as used herein means that a cassette containing a polynucleotide of interest is introduced into a plant or a plant cell and does not integrate into the genome of the plant or the plant cell or that a polypeptide is introduced into a plant or a plant cell.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway, et al., (1986) Biotechniques 4:320-334), electroporation (Riggs, et al., (1986) Proc. Natl. Acad. Sci. USA 83:5602-5606), Agrobacterium-mediated transformation (U.S. Pat. Nos. 5,563,055 and 5,981,840 and US Patent Publication 2017/0121722), direct gene transfer (Paszkowski, et al., (1984) EMBO J. 3:2717-2722) and ballistic particle acceleration (see, for example, U.S. Pat. Nos. 4,945,050; 5,879,918; 5,886,244 and 5,932,782; Tomes, et al., (1995) in Plant Cell, Tissue, and Organ Culture: Fundamental Methods, ed. Gamborg and Phillips, (Springer-Verlag, Berlin) and McCabe, et al., (1988) Biotechnology 6:923-926) and Led transformation (WO 00/28058). For potato transformation see, Tu, et al., (1998) Plant Molecular Biology 37:829-838 and Chong, et al., (2000) Transgenic Research 9:71-78. Additional transformation procedures can be found in Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); McCabe, et al., (1988) Bio/Technology 6:923-926 (soybean); Finer and McMullen, (1991) In Vitro Cell Dev. Biol. 27P:175-182 (soybean); Singh, et al., (1998) Theor. Appl. Genet. 96:319-324 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); U.S. Pat. Nos. 5,240,855; 5,322,783 and 5,324,646; Klein, et al., (1988) Plant Physiol. 91:440-444 (maize); Fromm, et al., (1990) Biotechnology 8:833-839 (maize); Hooykaas-Van Slogteren, et al., (1984) Nature (London) 311:763-764; U.S. Pat. No. 5,736,369 (cereals); Bytebier, et al., (1987) Proc. Natl. Acad. Sci. USA 84:5345-5349 (Liliaceae); De Wet, et al., (1985) in The Experimental Manipulation of Ovule Tissues, ed. Chapman, et al., (Longman, New York), pp. 197-209 (pollen); Kaeppler, et al., (1990) Plant Cell Reports 9:415-418 and Kaeppler, et al., (1992) Theor. Appl. Genet. 84:560-566 (whisker-mediated transformation); D 'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotechnology 14:745-750 (maize via Agrobacterium tumefaciens).

In specific aspects, the cassette can be provided to a plant or a plant cell using a variety of transient transformation methods. Such transient transformation methods include, but are not limited to, the introduction of a cassette containing a polynucleotide of interest or variants and fragments thereof directly into a plant or a plant cell or the introduction of a polypeptide transcript of interest into a plant or a plant cell. Such methods include, for example, microinjection or particle bombardment. See, for example, Crossway, et al., (1986) Mol. Gen. Genet. 202:179-185; Nomura, et al., (1986) Plant Sci. 44:53-58; Hepler, et al., (1994) Proc. Natl. Acad. Sci. 91:2176-2180 and Hush, et al., (1994) The Journal of Cell Science 107:775-784. Alternatively, a cassette containing a polynucleotide of interest can be transiently transformed into a plant or a plant cell using techniques known in the art. Such techniques include viral vector systems and the precipitation of the polynucleotide in a manner that precludes subsequent release of the DNA. Thus, transcription from the particle-bound DNA can occur, but the frequency with which it is released to become integrated into the genome is greatly reduced. Such methods include the use of particles coated with polyethyleneimine (PEI; Sigma #P3143).

Methods are known in the art for the targeted insertion of a cassette containing a polynucleotide of interest at a specific location in a plant genome. In one embodiment, the insertion of a cassette containing a polynucleotide of interest at a desired genomic location is achieved using a site-specific recombination system. See, for example, WO 1999/25821, WO 1999/25854, WO 1999/25840, WO 1999/25855 and WO 1999/25853. Briefly, a polynucleotide of interest can be contained in a transfer cassette flanked by two non-identical recombination sites. The transfer cassette is introduced into a plant or a plant cell that has a target site which is flanked by two non-identical recombination sites that correspond to the sites of the transfer cassette stably incorporated into its genome. An appropriate recombinase is provided and the transfer cassette is integrated at the target site. The polynucleotide of interest is thereby integrated at a specific chromosomal position in the plant genome.

Plant transformation vectors may be comprised of one or more DNA vectors needed for achieving plant transformation. For example, it is a common practice in the art to utilize plant transformation vectors that are comprised of more than one contiguous DNA segment. These vectors are often referred to in the art as “binary vectors”. Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector or cassette that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also present on this plasmid vector or cassette are sequences required for bacterial replication. The cis-acting sequences are arranged in a fashion which allows efficient transfer into plant cells and expression therein. For example, the selectable marker gene and the gene of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux, (2000) Trends in Plant Science 5:446-451). See also WO 2017/112006. Several types of Agrobacterium strains (e.g. LBA4404, GV3101, EHA101, EHA105, etc.) can be used for plant transformation. The second plasmid vector is not necessary for transforming the plants by other methods such as by microprojection, microinjection, electroporation, and polyethylene glycol.

In general, plant transformation methods involve transferring heterologous DNA into target plant cells (e.g. callus, undifferentiated callus, immature and mature embryos, immature zygotic embryo, immature and mature cotyledon, embryonic axis, suspension culture cells, protoplasts, leaf, leaf cells, root cells, phloem cells and pollen). Following integration of heterologous foreign DNA into plant cells, one then applies a maximum threshold level of appropriate selection (depending on the selectable marker gene) in the medium to kill the untransformed cells and separate and proliferate the putatively transformed cells that survive from this selection treatment by transferring regularly to a fresh medium. By continuous passage and challenge with appropriate selection, one identifies and proliferates the cells that are transformed with the cassette containing a gene of interest. Molecular and biochemical methods can then be used to confirm the presence of the integrated heterologous gene of interest into the genome of the transgenic plant.

Explants are typically transferred to a fresh supply of the same medium and cultured routinely. A general description of the techniques and methods for generating transgenic plants are found in Ayres and Park, (1994) Critical Reviews in Plant Science 13:219-239 and Bommineni and Jauhar, (1997) Maydica 42:107-120. Subsequently, the transformed cells are differentiated into shoots after placing on regeneration medium supplemented with a maximum threshold level of selecting agent. The shoots are then transferred to a selective rooting medium for recovering a rooted shoot or a plantlet. The transgenic plantlet then grows into a mature plant and produces fertile seeds (e.g., Hiei, et al., (1994) The Plant Journal 6:271-282; Ishida, et al., (1996) Nature Biotechnology 14:745-750).

The genes of interest may be provided to a plant or a plant cell by contacting the plant or the plant cell with a virus or viral nucleic acids. Generally, such methods involve incorporating the cassette containing a nucleotide of interest within a viral DNA or RNA molecule. Methods for providing plants with cassettes containing nucleotide constructs and producing the encoded proteins in the plants, which involve viral DNA or RNA molecules, are known in the art. See, for example, U.S. Pat. Nos. 5,889,191; 5,889,190; 5,866,785; 5,589,367 and 5,316,931.

Methods for transformation of chloroplasts are known in the art. See, for example, Svab, et al., (1990) Proc. Natl. Acad. Sci. USA 87:8526-8530; Svab and Maliga, (1993) Proc. Natl. Acad. Sci. USA 90:913-917; Svab and Maliga, (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported by McBride, et al., (1994) Proc. Natl. Acad. Sci. USA 91:7301-7305.

The gene of interest can be introduced into the genome of a plant or a plant cell using genome editing technologies, or a previously introduced gene of interest may be edited using genome editing technologies. For example, a gene of interest can be introduced into a desired location in the genome of a plant through the use of double-stranded break technologies such as TALENs, meganucleases, zinc finger nucleases, CRISPR-Cas, and the like. For example, a gene of interest can be introduced into a desired location in a genome using a CRISPR-Cas system, for the purpose of site-specific insertion. The desired location in a plant genome can be any desired target site for insertion, such as a genomic region amenable for breeding or may be a target site located in a genomic window with an existing trait of interest. Existing traits of interest could be either an endogenous trait or a previously introduced trait.

In some aspects, where a gene of interest or a fusion polynucleotide of the gene of interest has previously been introduced into a genome, genome editing technologies may be used to alter or modify the introduced polynucleotide sequence. Site specific modifications that can be introduced into a gene of interest include those produced using any method for introducing site specific modification, including, but not limited to, through the use of gene repair oligonucleotides (e.g. US Publication 2013/0019349), or through the use of double-stranded break technologies such as TALENs, meganucleases, zinc finger nucleases, CRISPR-Cas, and the like. Such technologies can be used to modify the previously introduced gene of interest through the insertion, deletion or substitution of nucleotides within the introduced polynucleotide. Alternatively, double-stranded break technologies can be used to add additional genes of interest to the introduced gene of interest. Additional sequences that may be added include, additional expression elements, such as enhancer and promoter sequences. Genome editing technologies may be used to position additional genes of interest in close proximity to the gene of interest within the genome of a plant, in order to generate molecular stacks of genes of interest.

Following introduction of heterologous foreign DNA into plant cells, the transformation or integration of a heterologous gene into the plant genome is confirmed by various methods such as analysis of nucleic acids, proteins, and metabolites associated with the integrated gene.

PCR analysis is a rapid method to screen transformed cells, tissue or shoots for the presence of an incorporated gene before transplanting into the soil (Sambrook and Russell, (2001) Molecular Cloning: A Laboratory Manual. Cold Spring Harbor Laboratory Press, Cold Spring Harbor, NY). PCR is carried out using oligonucleotide primers specific to the gene of interest or Agrobacterium vector background.

Plant transformation may be confirmed by Southern blot analysis of genomic DNA (Sambrook and Russell, (2001) supra). In Northern blot analysis, RNA is isolated from specific tissues of a transformant, fractionated in a formaldehyde agarose gel, and blotted onto a nylon filter according to standard procedures that are routinely used in the art (Sambrook and Russell, (2001) supra). Expression of RNA encoded by a gene of interest is then tested by hybridizing the filter to a radioactive probe derived from the gene of interest, by methods known in the art (Sambrook and Russell, (2001) supra). Western blot, biochemical assays and the like may be carried out on the transgenic plants to confirm the presence of the protein encoded by a gene of interest by standard procedures (Sambrook and Russell, 2001, supra) using antibodies that bind to one or more epitopes present on the polypeptide of interest.

Transgenic plants or transgenic plant cells to be tested in the methods of the disclosure for detecting the impacts on plant health attributable to the expression of one or more agronomically important polypeptide of interest in the transgenic plant or the transgenic plant cell may comprise a stack of one or more polynucleotides of interest, such as for example polynucleotides or fusion polynucleotides with one or more additional polynucleotides resulting in the production or suppression of multiple polypeptide sequences. Transgenic plants or transgenic plant cells comprising stacks of polynucleotide sequences can be obtained by either or both of traditional breeding methods or through genetic engineering methods. These methods include, but are not limited to, breeding individual lines each comprising a polynucleotide of interest, transforming a transgenic plant or transgenic plant cell comprising a gene of interest disclosed herein with a subsequent different gene of interest and co-transformation of genes of interest into a single plant cell. As used herein, the term “stacked” includes having multiple traits or genes of interest present in the same plant or plant cell (i.e., in the case of two traits present in the same plant or plant cell, both traits are incorporated into the nuclear genome, one trait is incorporated into the nuclear genome and one trait is incorporated into the genome of a plastid or both traits are incorporated into the genome of a plastid). In one non-limiting example, “stacked traits” comprise a molecular stack where the sequences are physically adjacent to each other.

Co-transformation of genes can be carried out using single transformation vectors comprising multiple genes or genes carried separately on multiple vectors. If the genes of interest are stacked by genetically transforming the plants, the polynucleotide sequences of interest can be combined at any time and in any order. The traits can be introduced simultaneously in a co-transformation protocol with the polynucleotides of interest provided by any combination of transformation cassettes. For example, if two genes of interest will be introduced, the two genes of interest can be contained in separate transformation cassettes (trans) or contained on the same transformation cassette (cis). Expression of the genes of interest can be driven by the same promoter or by different promoters. In certain cases, it may be desirable to introduce a transformation cassette that will suppress the expression of the polynucleotide of interest. This may be combined with any combination of other suppression cassettes or overexpression cassettes to generate the desired combination of traits in a plant or a plant cell. It is further recognized that polynucleotide sequences can be stacked at a desired genomic location using a site-specific recombination system. See, for example, WO 1999/25821, WO 1999/25854, WO 1999/25840, WO 1999/25855 and WO 1999/25853, all of which are herein incorporated by reference.

Genes of interest for testing in the methods of the disclosure are reflective of the commercial markets and interests of those involved in the development of a crop. Crops and markets of interest change, and as developing nations open world markets, new crops and technologies will also emerge. In addition, as our understanding of agronomic traits and characteristics such as yield and heterosis increases, the choice of genes for transformation will change accordingly. Categories of genes of interest, that can be tested in the methods of the disclosure, include, for example, genes encoding important traits for agronomics, insect resistance, disease resistance, herbicide resistance, sterility, grain characteristics, and production of commercial products such as, fine chemicals and pharmaceuticals. Other genes of interest that can be tested in the methods of the disclosure include, for example, those genes involved in information, such as zinc fingers, those involved in communication, such as kinases, and those involved in housekeeping, such as heat shock proteins.

Multiple genes of interest can be tested in the methods of the disclosure, for example insect resistance traits can be stacked with one or more additional input traits (e.g., herbicide resistance, fungal resistance, virus resistance, stress tolerance, disease resistance, male sterility, stalk strength, and the like) or output traits (e.g., increased yield, modified starches, improved oil profile, balanced amino acids, high lysine or methionine, increased digestibility, improved fiber quality, drought resistance, and the like). Thus, the methods of the disclosure can be used to detect the impacts on plant health of a complete agronomic package of improved crop quality with the ability to flexibly and cost effectively control any number of agronomic pests.

As used herein, “trait” refers to a physiological, morphological, biochemical, or physical characteristic of a plant or particular plant material or plant cell which is conferred by a native gene or genes or a heterologous gene or genes of interest. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring uptake of carbon dioxide, or by the observation of the expression level of a gene or genes, e.g., by employing Northern analysis, RT-PCR, microarray gene expression assays, or reporter gene expression systems, or by agricultural observations such as stress tolerance, yield, or pathogen tolerance. An “enhanced trait” includes improved or enhanced water use efficiency or drought tolerance, osmotic stress tolerance, high salinity stress tolerance, heat stress tolerance, enhanced cold tolerance, including cold germination tolerance, increased yield, enhanced nitrogen use efficiency, early plant growth and development, late plant growth and development, enhanced seed protein production, and enhanced seed oil production. The genes of interest imparting these enhanced traits can be tested in the method of the disclosure.

Genes affecting various changes in phenotype of a plant can be tested in the methods of the disclosure including, modifying the oil content such as levels and types of oils, saturated and unsaturated, the fatty acid composition, altering the amino acid content such as quality and quantity of essential amino acids, the starch content, cellulose starch content, or the carbohydrate content, protein content, altering nutrient metabolism, altering a metabolic pathway, altering pathogen defense mechanisms, altering kernel size, altering sucrose loading, and the like. The genes of interest to be tested in the methods of the disclosure may also be involved in regulating the influx of nutrients, and in regulating expression of phytate genes particularly to lower phytate levels in the seed.

These genes of interest can be modified by genetic alteration in addition to using traditional breeding methods for such modifications and retested in the methods of the disclosure. Modifications that can be tested in the methods of the disclosure include increasing content of oleic acid, saturated and unsaturated oils, increasing levels of lysine and sulfur, providing essential amino acids, and also modification of starch. Hordothionin protein modifications that can be tested in the methods of the disclosure are described in U.S. Pat. Nos. 5,703,049, 5,885,801, 5,885,802, and 5,990,389, herein incorporated by reference. Another example of a gene of interest that can be tested in the methods of the disclosure is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the genes of interest can be tested in the methods of the disclosure and can be made by site-directed mutagenesis to increase the level of preselected amino acids in the encoded polypeptide. For example, methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference) could be used. Other agronomically important genes encode latex, Floury 2, growth factors, seed storage factors, and transcription factors.

Insect resistance genes that encode resistance to pests that have great yield drag such as rootworm, cutworm, European Corn Borer, and the like can be tested in the methods of the disclosure. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109). Other non-limiting examples of Bacillus thuringiensis genes of interest that can be tested in the methods of the disclosure are those of the following patents and patent applications: U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849 and WO 1991/14778; WO 1999/31248; WO 2001/12731; WO 1999/24581 and WO 1997/40162.

Other non-limiting examples of genes of interest encoding insecticidal proteins that can be tested in the methods of the disclosure include those from Pseudomonas sp. such as PSEEN3174 (Monalysin, (2011) PLoS Pathogens, 7:1-13), from Pseudomonas protegens strain CHAO and Pf-5 (previously fluorescens) (Pechy-Tarr, (2008) Environmental Microbiology 10:2368-2386: GenBank Accession No. EU400157); from Pseudomonas taiwanensis (Liu, et al., (2010) J. Agric. Food Chem. 58:12343-12349) and from Pseudomonas pseudoalcaligenes (Zhang, et al., (2009) Annals of Microbiology 59:45-50 and Li, et al., (2007) Plant Cell Tiss. Organ Cult. 89:159-168); insecticidal proteins from Photorhabdus sp. and Xenorhabdus sp. (Hinchliffe, et al., (2010) The Open Toxinology Journal 3:101-118 and Morgan, et al., (2001) Applied and Envir. Micro. 67:2062-2069), U.S. Pat. Nos. 6,048,838, and 6,379,946; a PIP-1 polypeptide of US Patent Publication US20140007292; an AfIP-1A and/or AfIP-1B polypeptide of US Patent Publication US20140033361; a PHI-4 polypeptide of US Patent Publication US20140274885 and US20160040184; a PIP-47 polypeptide of PCT Publication Number WO2015/023846, a PIP-72 polypeptide of PCT Publication Number WO2015/038734; a PtIP-50 polypeptide and a PtIP-65 polypeptide of PCT Publication Number WO2015/120270; a PtIP-83 polypeptide of PCT Publication Number WO2015/120276; a PtIP-96 polypeptide of PCT Serial Number PCT/US15/55502; an IPD079 polypeptide of U.S. Ser. No. 62/201,977; an IPD082 polypeptide of U.S. Ser. No. 62/269,482, and 6-endotoxins including, but not limited to, the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry50, Cry51, Cry52, Cry53, Cry 54, Cry55, Cry56, Cry57, Cry58, Cry59, Cry60, Cry61, Cry62, Cry63, Cry64, Cry65, Cry66, Cry67, Cry68, Cry69, Cry70, Cry71, and Cry 72 classes of δ-endotoxin genes and the B. thuringiensis cytolytic Cyt1 and Cyt2 genes. Members of these classes of B. thuringiensis insecticidal proteins well known to one skilled in the art (see, Crickmore, et al., “Bacillus thuringiensis toxin nomenclature” (2011), at lifesci.sussex.ac.uk/home/Neil Crickmore/Bt/which can be accessed on the world-wide web using the “www” prefix).

Examples of δ-endotoxin genes of interest that can be tested in the methods of the disclosure also include but are not limited to those expressing Cry1A proteins of U.S. Pat. Nos. 5,880,275 and 7,858,849; a DIG-3 or DIG-11 toxin (N-terminal deletion of α-helix 1 and/or α-helix 2 variants of Cry proteins such as Cry1A) of U.S. Pat. Nos. 8,304,604 and 8,304,605, Cry1B of U.S. patent application Ser. No. 10/525,318; Cry1C of U.S. Pat. No. 6,033,874; Cry1F of U.S. Pat. Nos. 5,188,960, 6,218,188; Cry1A/F chimeras of U.S. Pat. Nos. 7,070,982; 6,962,705 and 6,713,063); a Cry2 protein such as Cry2Ab protein of U.S. Pat. No. 7,064,249); a Cry3A protein including but not limited to an engineered hybrid insecticidal protein (eHIP) created by fusing unique combinations of variable regions and conserved blocks of at least two different Cry proteins (US Patent Application Publication Number 2010/0017914); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760; a Cry9 protein such as such as members of the Cry9A, Cry9B, Cry9C, Cry9D, Cry9E, and Cry9F families; a Cry15 protein of Naimov, et al., (2008) Applied and Environmental Microbiology 74:7145-7151; a Cry22, a Cry34Ab1 protein of U.S. Pat. Nos. 6,127,180, 6,624,145 and 6,340,593; a CryET33 and CryET34 protein of U.S. Pat. Nos. 6,248,535, 6,326,351, 6,399,330, 6,949,626, 7,385,107 and 7,504,229; a CryET33 and CryET34 homologs of US Patent Publication Number 2006/0191034, 2012/0278954, and PCT Publication Number WO 2012/139004; a Cry35Ab1 protein of U.S. Pat. Nos. 6,083,499, 6,548,291 and 6,340,593; a Cry46 protein, a Cry 51 protein, a Cry binary toxin; a TIC901 or related toxin; TIC807 of US 2008/0295207; ET29, ET37, TIC809, TIC810, TIC812, TIC127, TIC128 of PCT US 2006/033867; AXMI-027, AXMI-036, and AXMI-038 of U.S. Pat. No. 8,236,757; AXMI-031, AXMI-039, AXMI-040, AXMI-049 of U.S. Pat. No. 7,923,602; AXMI-018, AXMI-020, and AXMI-021 of WO 2006/083891; AXMI-010 of WO 2005/038032; AXMI-003 of WO 2005/021585; AXMI-008 of US 2004/0250311; AXMI-006 of US 2004/0216186; AXMI-007 of US 2004/0210965; AXMI-009 of US 2004/0210964; AXMI-014 of US 2004/0197917; AXMI-004 of US 2004/0197916; AXMI-028 and AXMI-029 of WO 2006/119457; AXMI-007, AXMI-008, AXMI-0080rf2, AXMI-009, AXMI-014 and AXMI-004 of WO 2004/074462; AXMI-150 of U.S. Pat. No. 8,084,416; AXMI-205 of US20110023184; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063, and AXMI-064 of US 2011/0263488; AXMI-R1 and related proteins of US 2010/0197592; AXMI221Z, AXMI222z, AXMI223z, AXMI224z and AXMI225z of WO 2011/103248; AXMI218, AXMI219, AXMI220, AXMI226, AXMI227, AXMI228, AXMI229, AXMI230, and AXMI231 of WO11/103247; AXMI-115, AXMI-113, AXMI-005, AXMI-163 and AXMI-184 of U.S. Pat. No. 8,334,431; AXMI-001, AXMI-002, AXMI-030, AXMI-035, and AXMI-045 of US 2010/0298211; AXMI-066 and AXMI-076 of US2009/0144852; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of US 2010/0005543; and Cry proteins such as Cry1A and Cry3A having modified proteolytic sites of U.S. Pat. No. 8,319,019; and a Cry1Ac, Cry2Aa and Cry1Ca toxin protein from Bacillus thuringiensis strain VBTS 2528 of US Patent Application Publication Number 2011/0064710. Other Cry proteins that can be tested in the methods of the disclosure are well known to one skilled in the art (see, Crickmore, et al., “Bacillus thuringiensis toxin nomenclature” (2011), at lifesci.sussex.ac.uk/home/Neil_CrickmoreSt/which can be accessed on the world-wide web using the “www” prefix).

Combinations of genes of interest expressing pesticidal proteins can be tested in the methods of the disclosure such as Vip3Ab & Cry1Fa (US2012/0317682), Cry1BE & Cry1F (US2012/0311746), Cry1CA & Cry1AB (US2012/0311745), Cry1F & CryCa (US2012/0317681), Cry1DA & Cry1BE (US2012/0331590), Cry1DA & Cry1Fa (US2012/0331589), Cry1AB & Cry1BE (US2012/0324606), and Cry1Fa & Cry2Aa, Cry1I or Cry1E (US2012/0324605). Insecticidal lipases including lipid acyl hydrolases of U.S. Pat. No. 7,491,869, and cholesterol oxidases such as from Streptomyces (Purcell et al. (1993) Biochem Biophys Res Commun 15:1406-1413) can also be tested in the methods of the disclosure. Pesticidal proteins that can be tested in the methods of the disclosure also include VIP (vegetative insecticidal proteins) toxins of U.S. Pat. Nos. 5,877,012, 6,107,279, 6,137,033, 7,244,820, 7,615,686, and 8,237,020. Other VIP proteins that can be tested in the methods of the disclosure are well known to one skilled in the art (see, lifesci.sussex.ac.uk/home/Neil_CrickmoreSt/vip.html which can be accessed on the world-wide web using the “www” prefix). Pesticidal proteins that can be tested in the methods of the disclosure also include toxin complex (TC) proteins, obtainable from organisms such as Xenorhabdus, Photorhabdus and Paenibacillus (see, U.S. Pat. Nos. 7,491,698 and 8,084,418). Pesticidal proteins that can be tested in the methods of the disclosure also include spider, snake and scorpion venom proteins. Examples of spider venom peptides that can be tested in the methods of the disclosure include but are not limited to lycotoxin-1 peptides and mutants thereof (U.S. Pat. No. 8,334,366).

Further transgenes that confer resistance to insects that can be tested in the methods of the disclosure may down-regulate expression of target genes in insect pest species by interfering ribonucleic acid (RNA) molecules through RNA interference. RNA interference refers to the process of sequence-specific post-transcriptional gene silencing in animals mediated by short interfering RNAs (siRNAs) (Fire, et al., (1998) Nature 391:806). RNAi transgenes that can be tested in the methods of the disclosure may include but are not limited to expression of dsRNA, siRNA, miRNA, iRNA, antisense RNA, or sense RNA molecules that down-regulate expression of target genes in insect pests.

RNAi transgenes targeting the vacuolar ATPase H subunit, useful for controlling a coleopteran pest population and infestation as described in US Patent Application Publication 2012/0198586 can be tested in the methods of the disclosure. PCT Publication WO 2012/055982 describes ribonucleic acid (RNA or double stranded RNA) that inhibits or down regulates the expression of a target gene that encodes: an insect ribosomal protein such as the ribosomal protein L19, the ribosomal protein L40 or the ribosomal protein S27A; an insect proteasome subunit such as the Rpn6 protein, the Pros 25, the Rpn2 protein, the proteasome beta 1 subunit protein or the Pros beta 2 protein; an insect β-coatomer of the COPI vesicle, the γ-coatomer of the COPI vesicle, the β′-coatomer protein or the ζ-coatomer of the COPI vesicle; an insect Tetraspanine 2 A protein which is a putative transmembrane domain protein; an insect protein belonging to the actin family such as Actin 5C; an insect ubiquitin-5E protein; an insect Sec23 protein which is a GTPase activator involved in intracellular protein transport; an insect crinkled protein which is an unconventional myosin which is involved in motor activity; an insect crooked neck protein which is involved in the regulation of nuclear alternative mRNA splicing; an insect vacuolar H+-ATPase G-subunit protein and an insect Tbp-1 such as Tat-binding protein can be tested in the methods of the disclosure. PCT publication WO 2007/035650 describes ribonucleic acid (RNA or double stranded RNA) that inhibits or down regulates the expression of a target gene that encodes Snf7 that can be tested in the methods of the disclosure. US Patent Application publication 2011/0054007 describes polynucleotide silencing elements targeting RPS10 that can be tested in the methods of the disclosure. US Patent Application publication 2014/0275208 and US2015/0257389 describes polynucleotide silencing elements targeting RyanR and PAT3 that can be tested in the methods of the disclosure. PCT publications WO/2016/138106, WO 2016/060911, WO 2016/060912, WO 2016/060913, and WO 2016/060914 describe polynucleotide silencing elements targeting COPI coatomer subunit nucleic acid molecules that confer resistance to Coleopteran and Hemipteran pests that can be tested in the methods of the disclosure. US Patent Application Publications 2012/029750, US 20120297501, and 2012/0322660 describe interfering ribonucleic acids (RNA or double stranded RNA) that functions upon uptake by an insect pest species to down-regulate expression of a target gene in said insect pest, wherein the RNA comprises at least one silencing element wherein the silencing element is a region of double-stranded RNA comprising annealed complementary strands, one strand of which comprises or consists of a sequence of nucleotides which is at least partially complementary to a target nucleotide sequence within the target gene that can be tested in the methods of the disclosure. US Patent Application Publication 2012/0164205 describe potential targets for interfering double stranded ribonucleic acids for inhibiting invertebrate pests including: a Chd3 Homologous Sequence, a Beta-Tubulin Homologous Sequence, a 40 kDa V-ATPase Homologous Sequence, a EFla Homologous Sequence, a 26S Proteosome Subunit p28 Homologous Sequence, a Juvenile Hormone Epoxide Hydrolase Homologous Sequence, a Swelling Dependent Chloride Channel Protein Homologous Sequence, a Glucose-6-Phosphate 1-Dehydrogenase Protein Homologous Sequence, an Act42A Protein Homologous Sequence, a ADP-Ribosylation Factor 1 Homologous Sequence, a Transcription Factor IIB Protein Homologous Sequence, a Chitinase Homologous Sequences, a Ubiquitin Conjugating Enzyme Homologous Sequence, a Glyceraldehyde-3-Phosphate Dehydrogenase Homologous Sequence, an Ubiquitin B Homologous Sequence, a Juvenile Hormone Esterase Homolog, and an Alpha Tubuliln Homologous Sequence that can be tested in the methods of the disclosure.

Genes of interest encoding disease resistance traits include detoxification genes, such as those against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089) can be tested in the methods of the disclosure.

Herbicide resistance traits that can be tested in the methods of the disclosure may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), glyphosate (e.g., the EPSPS gene and the GAT gene; see, for example, U.S. Publication No. 20040082770 and WO 03/092360) or other such genes known in the art.

Sterility genes can also be tested in the methods of the disclosure. Examples of genes used in such ways that can be tested in the methods of the disclosure include male tissue-preferred genes and genes with male sterility phenotypes such as QM, described in U.S. Pat. No. 5,583,210. Other genes that can be tested in the methods of the disclosure include kinases and those encoding compounds toxic to either male or female gametophytic development.

As used herein, the term “morphogenic gene” means a gene that when ectopically expressed stimulates formation of a somatically-derived structure that can produce a plant. More precisely, ectopic expression of the morphogenic gene stimulates the de novo formation of a somatic embryo or an organogenic structure, such as a shoot meristem, that can produce a plant. This stimulated de novo formation occurs either in the cell in which the morphogenic gene is expressed, or in a neighboring cell. A morphogenic gene can be a transcription factor that regulates expression of other genes, or a gene that influences hormone levels in a plant tissue, both of which can stimulate morphogenic changes.

A morphogenic gene is involved in plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof, such as WUS/WOX genes (WUS1, WUS2, WUS3, WOX2A, WOX4, WOX5, or WOX9) see U.S. Pat. Nos. 7,348,468 and 7,256,322 and United States Patent Application publications 20170121722 and 20070271628; Laux et al. (1996) Development 122:87-96; and Mayer et al. (1998) Cell 95:805-815; van der Graaff et al., 2009, Genome Biology 10:248; Dolzblasz et al., 2016, Mol. Plant 19:1028-39. Modulation of WUS/WOX is expected to modulate plant and/or plant tissue phenotype including plant metabolism, organ development, stem cell development, cell growth stimulation, organogenesis, somatic embryogenesis initiation, accelerated somatic embryo maturation, initiation and/or development of the apical meristem, initiation and/or development of shoot meristem, or a combination thereof. Expression of Arabidopsis WUS can induce stem cells in vegetative tissues, which can differentiate into somatic embryos (Zuo, et al. (2002) Plant J 30:349-359). Also of interest in this regard would be a MYB118 gene (see U.S. Pat. No. 7,148,402), MYB115 gene (see Wang et al. (2008) Cell Research 224-235), a BABYBOOM gene (BBM; see Boutilier et al. (2002) Plant Cell 14:1737-1749), or a CLAVATA gene (see, for example, U.S. Pat. No. 7,179,963).

Other morphogenic genes useful in the present disclosure include, but are not limited to, LEC1 (Lotan et al., 1998, Cell 93:1195-1205), LEC2 (Stone et al., 2008, PNAS 105:3151-3156; Belide et al., 2013, Plant Cell Tiss. Organ Cult 113:543-553), KN1/STM (Sinha et al., 1993. Genes Dev 7:787-795), the IPT gene from Agrobacterium (Ebinuma and Komamine, 2001, In vitro Cell. Dev Biol—Plant 37:103-113), MONOPTEROS-DELTA (Ckurshumova et al., 2014, New Phytol. 204:556-566), the Agrobacterium AV-6b gene (Wabiko and Minemura 1996, Plant Physiol. 112:939-951), the combination of the Agrobacterium IAA-h and IAA-m genes (Endo et al., 2002, Plant Cell Rep., 20:923-928), the Arabidopsis SERK gene (Hecht et al., 2001, Plant Physiol. 127:803-816), the Arabidopsis AGL15 gene (Harding et al., 2003, Plant Physiol. 133:653-663).

As used herein, the term “transcription factor” means a protein that controls the rate of transcription of specific genes by binding to the DNA sequence of the promoter and either up-regulating or down-regulating expression. Examples of transcription factors, which are also morphogenic genes, include members of the AP2/EREBP family (including the BBM (ODP2), plethora and aintegumenta sub-families, CAAT-box binding proteins such as LEC1 and HAP3, and members of the MYB, bHLH, NAC, MADS, bZIP and WRKY families.

A morphogenic gene may be stably incorporated into the genome of a plant or it may be transiently expressed.

The use of the term “nucleotide construct” or “expression cassette” as used herein is not intended to limit the disclosure to nucleotide constructs or expression cassettes comprising DNA. Those of ordinary skill in the art will recognize that nucleotide constructs and expression cassettes, particularly polynucleotides and oligonucleotides composed of ribonucleotides and combinations of ribonucleotides and deoxyribonucleotides, may also be employed in the methods disclosed herein. The nucleotide constructs, expression cassettes, nucleic acids, nucleotide sequences, and genes of interest useful in the methods of the disclosure additionally encompass all complementary forms of such constructs, cassettes, genes, molecules, and sequences. Further, the nucleotide constructs, expression cassettes, nucleic acids, nucleotide molecules, nucleotide sequences, and genes of interest useful in the methods of the disclosure encompass all nucleotide constructs, expression cassettes, genes, molecules, and sequences which can be employed in the methods of the disclosure for transforming plants including, but not limited to, those comprised of deoxyribonucleotides, ribonucleotides, and combinations thereof. Such deoxyribonucleotides and ribonucleotides include both naturally occurring molecules and synthetic analogues. The nucleotide constructs, expression cassettes, nucleic acids, nucleotide sequences, and genes of interest useful in the methods of the disclosure also encompass all forms of nucleotide constructs and expression cassettes including, but not limited to, single-stranded forms, double-stranded forms, hairpins, stem-and-loop structures and the like.

Genes of interest and neutral control genes to be tested in the methods of the disclosure are provided in DNA cassettes or constructs for expression in a plant or plant cell. The cassette or construct will include 5′ and 3′ regulatory sequences operably linked to a gene of interest or a neutral control gene. The term “operably linked” as used herein refers to a functional linkage or association between a regulatory sequence and a second sequence so that the function of one is affected by the other (e.g., nucleic acid sequences being linked are contiguous and where necessary join two protein coding regions in the same reading frame). For example, a promoter is operably linked with a gene of interest or a neutral control gene when it is capable of affecting the expression of that gene of interest or that neutral control gene (i.e., that the gene of interest or neutral control gene is under the transcriptional control of the promoter). Coding sequences can be operably linked to regulatory sequences in sense or antisense orientation. As used herein, “antisense orientation” includes reference to a polynucleotide sequence that is operably linked to a promoter in an orientation where the antisense strand is transcribed. The antisense strand is sufficiently complementary to an endogenous transcription product such that translation of the endogenous transcription product is often inhibited. When the regulatory sequence is a promoter, the promoter sequence initiates and mediates transcription of the DNA sequence corresponding to the second sequence. The cassette or construct may additionally contain at least one additional gene of interest to be cotransformed into the plant or plant cell. Alternatively, the additional gene(s) of interest can be provided on multiple DNA cassettes or constructs.

Such a DNA cassette or construct is provided with a plurality of restriction sites for insertion of a gene of interest to be under the transcriptional regulation of the regulatory sequences. The DNA cassette or construct will generally include in the 5′ to 3′ direction of transcription: a transcriptional and translational initiation region (i.e., a promoter), a gene of interest or a neutral control gene to be tested in the methods of the disclosure, and a transcriptional and translational termination region (i.e., termination region) functional in a plant or plant cell. The DNA cassette or construct may additionally contain selectable marker genes.

The transcriptional initiation region (i.e., the promoter) may be native, analogous, foreign or heterologous to the plant or plant cell and/or to the genes of interest or the neutral control genes to be tested in the methods of the disclosure. Additionally, the promoter may be the natural sequence or alternatively a synthetic sequence. The term “foreign” as used herein indicates that the promoter is not found in the plant or plant cell into which the promoter is introduced. Where the promoter is “foreign” or “heterologous” to the genes of interest or the neutral control genes, it is intended that the promoter is not the native or naturally occurring promoter for the operably linked genes of interest or neutral control genes to be tested in the methods of the disclosure. As used herein, a chimeric gene of interest or a chimeric neutral control gene to be tested in the methods of the disclosure comprises a coding sequence operably linked to a transcription initiation region that is heterologous to the gene of interest or the neutral control gene.

Genes of interest and neutral control genes to be tested in the methods of the disclosure can be operably linked to a suitable promoter. “Promoter” means a region of DNA that is upstream from the start of transcription and is involved in recognition and binding of RNA polymerase and other proteins to initiate transcription, either including or not including a 5′ UTR. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as from Agrobacterium or Rhizobium. Examples of promoters under developmental control include promoters that preferentially initiate transcription in certain tissues, such as leaves, roots, or seeds. Such promoters are referred to as “tissue preferred” promoters. Promoters which initiate transcription only in certain tissues are referred to as “tissue specific” promoters. A “cell type” specific promoter primarily drives expression in certain cell types in one or more organs, for example, vascular cells in roots or leaves. An “inducible” or “repressible” promoter can be a promoter which is under either environmental or exogenous control. Examples of environmental conditions that may affect transcription by inducible promoters include anaerobic conditions, or the presence of certain chemicals, or the presence of light. Alternatively, exogenous control of an inducible or repressible promoter can be affected by providing a suitable chemical or other agent that via interaction with target polypeptides result in induction or repression of the promoter. Tissue specific, tissue preferred, cell type specific, and inducible promoters constitute the class of “non-constitutive” promoters. A “constitutive” promoter is a promoter which is active under most conditions.

A number of promoters can be used in the practice of the methods of the disclosure. The promoters can be selected based on the desired outcome. The genes of interest and the neutral control genes can be combined with constitutive, tissue-preferred, inducible or other promoters for expression in the host organism. Suitable constitutive promoters for use in a plant or a plant cell include, for example, the core promoter of the Rsyn7 promoter and other constitutive promoters disclosed in WO 1999/43838 and U.S. Pat. No. 6,072,050; the core CaMV 35S promoter (Odell, et al., (1985) Nature 313:810-812); rice actin (McElroy, et al., (1990) Plant Cell 2:163-171); ubiquitin (Christensen, et al., (1989) Plant Mol. Biol. 12:619-632 and Christensen, et al., (1992) Plant Mol. Biol. 18:675-689); pEMU (Last, et al., (1991) Theor. Appl. Genet. 81:581-588); MAS (Velten, et al., (1984) EMBO J. 3:2723-2730); ALS promoter (U.S. Pat. No. 5,659,026) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; 6,177,611 and the AtUBQ10 promoter (Day, et. al., (1999) Plant Mol. Biol. 40:771-782; Norris S R et al (1993) Plant Mol Biol. 21(5):895-906).

Depending on the desired outcome, it may be beneficial to express the gene of interest or the neutral control gene from an inducible promoter. Of particular interest for regulating the expression of the genes of interest or the neutral control genes in plants or plant cells are wound-inducible promoters. Such wound-inducible promoters, may respond to damage caused by insect feeding, and include potato proteinase inhibitor (pin II) gene (Ryan, (1990) Ann. Rev. Phytopath. 28:425-449; Duan, et al., (1996) Nature Biotechnology 14:494-498); wun1 and wun2, U.S. Pat. No. 5,428,148; win1 and win2 (Stanford, et al., (1989) Mol. Gen. Genet. 215:200-208); systemin (McGurl, et al., (1992) Science 225:1570-1573); WIP1 (Rohmeier, et al., (1993) Plant Mol. Biol. 22:783-792; Eckelkamp, et al., (1993) FEBS Letters 323:73-76); MPI gene (Corderok, et al., (1994) Plant J. 6(2):141-150), and the like.

Additionally, pathogen-inducible promoters may be employed in the methods of the disclosure. Such pathogen-inducible promoters include those from pathogenesis-related proteins (PR proteins), which are induced following infection by a pathogen; e.g., PR proteins, SAR proteins, beta-1,3-glucanase, chitinase, etc. See, for example, Redolfi, et al., (1983) Neth. J. Plant Pathol. 89:245-254; Uknes, et al., (1992) Plant Cell 4: 645-656 and Van Loon, (1985) Plant Mol. Virol. 4:111-116. See also, WO 1999/43819. Of interest are promoters that are expressed locally at or near the site of pathogen infection. See, for example, Marineau, et al., (1987) Plant Mol. Biol. 9:335-342; Matton, et al., (1989) Molecular Plant-Microbe Interactions 2:325-331; Somsisch, et al., (1986) Proc. Natl. Acad. Sci. USA 83:2427-2430; Somsisch, et al., (1988) Mol. Gen. Genet. 2:93-98 and Yang, (1996) Proc. Natl. Acad. Sci. USA 93:14972-14977. See also, Chen, et al., (1996) Plant J. 10:955-966; Zhang, et al., (1994) Proc. Natl. Acad. Sci. USA 91:2507-2511; Warner, et al., (1993) Plant J. 3:191-201; Siebertz, et al., (1989) Plant Cell 1:961-968; U.S. Pat. No. 5,750,386 (nematode-inducible) and the references cited therein. Of particular interest is the inducible promoter for the maize PRms gene, whose expression is induced by the pathogen Fusarium moniliforme (see, for example, Cordero, et al., (1992) Physiol. Mol. Plant Path. 41:189-200).

Chemical-regulated promoters can be used to modulate the expression of a gene of interest or a neutral control gene in a plant or a plant cell through the application of an exogenous chemical regulator. Depending upon the objective, the promoter may be a chemical-inducible promoter, where application of the chemical induces gene expression or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-la promoter, which is activated by salicylic acid. Other chemical-regulated promoters of interest include steroid-responsive promoters (see, for example, glucocorticoid-inducible promoter disclosed in Schena, et al., (1991) Proc. Natl. Acad. Sci. USA 88:10421-10425 and McNellis, et al., (1998) Plant J. 14(2):247-257), tetracycline-inducible and tetracycline-repressible promoters (see, for example, Gatz, et al., (1991) Mol. Gen. Genet. 227:229-237 and U.S. Pat. Nos. 5,814,618 and 5,789,156, as well as sulfonylurea inducible promoters disclosed in U.S. Pat. No. 8,877,503).

Tissue-preferred promoters can be utilized to target expression of a gene of interest or a neutral control gene within a particular plant tissue. Tissue-preferred promoters include those discussed in Yamamoto, et al., (1997) Plant J. 12(2)255-265; Kawamata, et al., (1997) Plant Cell Physiol. 38(7):792-803; Hansen, et al., (1997) Mol. Gen Genet. 254(3):337-343; Russell, et al., (1997) Transgenic Res. 6(2):157-168; Rinehart, et al., (1996) Plant Physiol. 112(3):1331-1341; Van Camp, et al., (1996) Plant Physiol. 112(2):525-535; Canevascini, et al., (1996) Plant Physiol. 112(2):513-524; Yamamoto, et al., (1994) Plant Cell Physiol. 35(5):773-778; Lam, (1994) Results Probl. Cell Differ. 20:181-196; Orozco, et al., (1993) Plant Mol Biol. 23(6):1129-1138; Matsuoka, et al., (1993) Proc Natl. Acad. Sci. USA 90(20):9586-9590 and Guevara-Garcia, et al., (1993) Plant J. 4(3):495-505. Such promoters can be modified, if necessary, for weak expression.

Leaf-preferred promoters are known in the art and can be utilized in the methods of the disclosure. See, for example, Yamamoto, et al., (1997) Plant J. 12(2):255-265; Kwon, et al., (1994) Plant Physiol. 105:357-67; Yamamoto, et al., (1994) Plant Cell Physiol. 35(5):773-778; Gotor, et al., (1993) Plant J. 3:509-18; Orozco, et al., (1993) Plant Mol. Biol. 23(6):1129-1138 and Matsuoka, et al., (1993) Proc. Natl. Acad. Sci. USA 90(20):9586-9590.

Root-preferred or root-specific promoters are known and can be selected from the many available from the literature or isolated de novo from various compatible species for use in the methods of the disclosure. See, for example, Hire, et al., (1992) Plant Mol. Biol. 20(2):207-218 (soybean root-specific glutamine synthetase gene); Keller and Baumgartner, (1991) Plant Cell 3(10):1051-1061 (root-specific control element in the GRP 1.8 gene of French bean); Sanger, et al., (1990) Plant Mol. Biol. 14(3):433-443 (root-specific promoter of the mannopine synthase (MAS) gene of Agrobacterium tumefaciens) and Miao, et al., (1991) Plant Cell 3(1):11-22 (full-length cDNA clone encoding cytosolic glutamine synthetase (GS), which is expressed in roots and root nodules of soybean) for suitable root-preferred or root-specific promoters useful in the methods of the disclosure. See also, Bogusz, et al., (1990) Plant Cell 2(7):633-641, in which two root-specific promoters isolated from hemoglobin genes from the nitrogen-fixing nonlegume Parasponia andersonii and the related non-nitrogen-fixing nonlegume Trema tomentosa are described. Leach and Aoyagi, (1991) describe their analysis of the promoters of the highly expressed rolC and rolD root-inducing genes of Agrobacterium rhizogenes (see, Plant Science (Limerick) 79(1):69-76). Teeri, et al., (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene of interest (see, EMBO J. 8(2):343-350). The TR1′ gene fused to nptll (neomycin phosphotransferase II) showed similar characteristics. Additional root-preferred promoters include the VfENOD-GRP3 gene promoter (Kuster, et al., (1995) Plant Mol. Biol. 29(4):759-772) and rolB promoter (Capana, et al., (1994) Plant Mol. Biol. 25(4):681-691. See also, U.S. Pat. Nos. 5,837,876; 5,750,386; 5,633,363; 5,459,252; 5,401,836; 5,110,732 and 5,023,179. Arabidopsis thaliana root-preferred regulatory sequences are disclosed in US20130117883.

“Seed-preferred” promoters useful in the methods of the disclosure include both “seed-specific” promoters (those promoters active during seed development such as promoters of seed storage proteins) as well as “seed-germinating” promoters (those promoters active during seed germination). See, Thompson, et al., (1989) BioEssays 10:108. Such seed-preferred promoters include, but are not limited to, Cim1 (cytokinin-induced message); cZ19B1 (maize 19 kDa zein); and milps (myo-inositol-1-phosphate synthase) (see, U.S. Pat. No. 6,225,529). Gamma-zein and Glb-1 are endosperm-specific promoters. For dicots, seed-specific promoters include, but are not limited to, Kunitz trypsin inhibitor 3 (KTi3) (Jofuku and Goldberg, (1989) Plant Cell 1:1079-1101), bean β-phaseolin, napin, β-conglycinin, glycinin 1, soybean lectin, cruciferin, seed coat promoter from Arabidopsis, pBAN; the early seed promoters from Arabidopsis, p26, p63, and p63tr (U.S. Pat. Nos. 7,294,760 and 7,847,153), and the like. For monocots, seed-specific promoters include, but are not limited to, maize 15 kDa zein, 22 kDa zein, 27 kDa zein, g-zein, waxy, shrunken 1, shrunken 2, globulin 1, etc. See also, WO 2000/12733, where seed-preferred promoters from end1 and end2 genes are disclosed.

A promoter that has “preferred” expression in a particular tissue is expressed in that tissue to a greater degree than in at least one other plant tissue. Some tissue-preferred promoters show expression almost exclusively in the particular tissue.

Where low level expression is desired, weak promoters will be used. Generally, the term “weak promoter” as used herein refers to a promoter that drives expression of a coding sequence at a low level. By low level expression at levels of between about 1/1000 transcripts to about 1/100,000 transcripts to about 1/500,000 transcripts is intended. Alternatively, it is recognized that the term “weak promoters” also encompasses promoters that drive expression in only a few cells and not in others to give a total low level of expression. Where a promoter drives expression at unacceptably high levels, portions of the promoter sequence can be deleted or modified to decrease expression levels.

The above list of promoters is not meant to be limiting. Any appropriate promoter can be used in the methods of the disclosure.

In some aspects the DNA cassette or construct may also include a transcriptional enhancer sequence. As used herein, the term an “enhancer” refers to a DNA sequence which can stimulate promoter activity, and may be an innate element of the promoter or a heterologous element inserted to enhance the level or tissue-specificity of a promoter. Various enhancers are known in the art including for example, introns with gene expression enhancing properties in plants (US Patent Application Publication Number 2009/0144863, the ubiquitin intron (i.e., the maize ubiquitin intron 1 (see, for example, NCBI sequence S94464)), the omega enhancer or the omega prime enhancer (Gallie, et al., (1989) Molecular Biology of RNA ed. Cech (Liss, New York) 237-256 and Gallie, et al., (1987) Gene 60:217-25), the CaMV 35S enhancer (see, e.g., Benfey, et al., (1990) EMBO J. 9:1685-96) and the enhancers of U.S. Pat. No. 7,803,992 may also be used in the methods of the disclosure. The above list of transcriptional enhancers is not meant to be limiting. Any appropriate transcriptional enhancer can be used in the methods of the disclosure.

The transcriptional and translational termination region may be native with the transcriptional initiation region, may be native with the operably linked gene of interest or neutral control gene, may be native with the plant or plant cell or may be derived from another source (i.e., foreign or heterologous to the promoter, the gene of interest, the neutral control gene, the plant or plant cell or any combination thereof).

Convenient termination regions for use in the methods of the disclosure are available from the Ti-plasmid of A. tumefaciens, such as the octopine synthase and nopaline synthase termination regions. See also, Guerineau, et al., (1991) Mol. Gen. Genet. 262:141-144; Proudfoot, (1991) Cell 64:671-674; Sanfacon, et al., (1991) Genes Dev. 5:141-149; Mogen, et al., (1990) Plant Cell 2:1261-1272; Munroe, et al., (1990) Gene 91:151-158; Ballas, et al., (1989) Nucleic Acids Res. 17:7891-7903 and Joshi, et al., (1987) Nucleic Acid Res. 15:9627-9639 for additional terminators useful in the methods of the disclosure.

Where appropriate, a gene of interest or a neutral control gene may be optimized for increased expression in the plant or plant cell. Thus, the synthetic genes of interest and neutral control genes can be synthesized using plant-preferred codons for improved expression. See, for example, Campbell and Gowri, (1990) Plant Physiol. 92:1-11 for a discussion of host-preferred usage.

Additional sequence modifications are known to enhance gene expression in a plant or plant cell. These include elimination of sequences encoding spurious polyadenylation signals, exon-intron splice site signals, transposon-like repeats, and other well-characterized sequences that may be deleterious to gene expression. The GC content of the sequence may be adjusted to levels average for a given plant or plant cell, as calculated by reference to known genes expressed in the plant or plant cell. When possible, the sequence of a gene of interest or a neutral control gene is modified to avoid predicted hairpin secondary mRNA structures.

In preparing the expression cassette, the various DNA fragments may be manipulated to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells or tissues. Marker genes useful in the methods of the disclosure include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones and 2,4-dichlorophenoxyacetate (2,4-D). Additional examples of suitable selectable marker genes include, but are not limited to, genes encoding resistance to chloramphenicol (Herrera Estrella, et al., (1983) EMBO J. 2:987-992); methotrexate (Herrera Estrella, et al., (1983) Nature 303:209-213 and Meijer, et al., (1991) Plant Mol. Biol. 16:807-820); streptomycin (Jones, et al., (1987) Mol. Gen. Genet. 210:86-91); spectinomycin (Bretagne-Sagnard, et al., (1996) Transgenic Res. 5:131-137); bleomycin (Hille, et al., (1990) Plant Mol. Biol. 7:171-176); sulfonamide (Guerineau, et al., (1990) Plant Mol. Biol. 15:127-136); bromoxynil (Stalker, et al., (1988) Science 242:419-423); glyphosate (Shaw, et al., (1986) Science 233:478-481 and U.S. patent application Ser. Nos. 10/004,357 and 10/427,692); phosphinothricin (DeBlock, et al., (1987) EMBO J. 6:2513-2518). See generally, Yarranton, (1992) Curr. Opin. Biotech. 3:506-511; Christopherson, et al., (1992) Proc. Natl. Acad. Sci. USA 89:6314-6318; Yao, et al., (1992) Cell 71:63-72; Reznikoff, (1992) Mol. Microbiol. 6:2419-2422; Barkley, et al., (1980) in The Operon, pp. 177-220; Hu, et al., (1987) Cell 48:555-566; Brown, et al., (1987) Cell 49:603-612; Figge, et al., (1988) Cell 52:713-722; Deuschle, et al., (1989) Proc. Natl. Acad. Sci. USA 86:5400-5404; Fuerst, et al., (1989) Proc. Natl. Acad. Sci. USA 86:2549-2553; Deuschle, et al., (1990) Science 248:480-483; Gossen, (1993) Ph.D. Thesis, University of Heidelberg; Reines, et al., (1993) Proc. Natl. Acad. Sci. USA 90:1917-1921; Labow, et al., (1990) Mol. Cell. Biol. 10:3343-3356; Zambretti, et al., (1992) Proc. Natl. Acad. Sci. USA 89:3952-3956; Bairn, et al., (1991) Proc. Natl. Acad. Sci. USA 88:5072-5076; Wyborski, et al., (1991) Nucleic Acids Res. 19:4647-4653; Hillenand-Wissman, (1989) Topics Mol. Struc. Biol. 10:143-162; Degenkolb, et al., (1991) Antimicrob. Agents Chemother. 35:1591-1595; Kleinschnidt, et al., (1988) Biochemistry 27:1094-1104; Bonin, (1993) Ph.D. Thesis, University of Heidelberg; Gossen, et al., (1992) Proc. Natl. Acad. Sci. USA 89:5547-5551; Oliva, et al., (1992) Antimicrob. Agents Chemother. 36:913-919; Hlavka, et al., (1985) Handbook of Experimental Pharmacology, Vol. 78 (Springer-Verlag, Berlin) and Gill, et al., (1988) Nature 334:721-724 for selectable marker genes useful in the methods of the disclosure.

The above list of selectable marker genes is not meant to be limiting. Any selectable marker gene can be used in the methods of the disclosure.

The expression cassette will further comprise a neutral control gene. Non-limiting examples of neutral control genes useful in the methods of the disclosure include a chloramphenicol acetyl transferase (CAT) gene, a fluorescent protein (FP) gene, a phosphomannose isomerase (PMI) gene, a β-glucuronidase (GUS) gene, or a housekeeping gene.

Housekeeping genes useful as neutral control genes in the methods of the disclosure are typically constitutive genes that are required for the maintenance of basic cellular function, and are expressed in all cells of an organism under normal and patho-physiological conditions. Although some housekeeping genes are expressed at relatively constant levels in most non-pathological situations, other housekeeping genes may vary depending on experimental conditions and the expression of one or multiple housekeeping genes can be used as a reference point for the analysis of expression levels of other genes. Non-limiting examples of housekeeping genes useful in the methods of the disclosure include beta-tubulin, cyclophilin, actin, elongation factor 1-alpha (eflalpha), 18S rRNA, adenine phosphoribosyl transferase (aprt), and cytoplasmic ribosomal protein L2.

The above list of neutral control genes is not meant to be limiting. Any neutral control gene can be used in the methods of the disclosure.

The expression cassette may further comprise a reporter gene. Reporter genes useful in the methods of the disclosure include an ATP dependent luciferase gene (i.e. Firefly luciferase), an ATP independent luciferase (i.e. Renilla luciferase), a chloramphenicol acetyl transferase (CAT) gene, a fluorescent protein (FP) gene, a β-glucuronidase (GUS) gene, a β-galactosidase (GAL) gene, or an alkaline phosphatase gene.

The above list of reporter genes is not meant to be limiting. Any reporter gene can be used in the methods of the disclosure.

Fluorescent protein (FP) genes useful in the methods of the disclosure include GFP, EGFP, Emerald, Superfolder GFP, Azami Green, mWasabi, TagGFP, TurboGFP, AcGFP, ZsGreen, T-Sapphire, EBFP, EBFP2, Azurite, TagBFP, ECFP, mECFP, Cerulean, mTurquoise, CyPet, AmCyanl, Midori-Ishi Cyan, TagCFP, mTFP1 (Teal), EYFP, Topaz, Venus, mCitrine, YPet, TagYFP, PhiYFP, ZsYellowl, mBanana, Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, mRuby, mApple, mStrawberry, AsRed2, mRFP1, JRed, mCherry, HcRedl, mRaspberry, dKeima-Tandem, HcRed-Tandem, mPlum, or AQ143. Additional information about these fluorescent proteins is available at: microscopyu.com/techniques/fluorescence/introduction-to-fluorescent-proteins which can be accessed on the world-wide web using the “www” prefix.

The above list of fluorescent protein (FP) genes is not meant to be limiting. Any fluorescent protein (FP) gene can be used in the methods of the disclosure.

### EXAMPLES

### Example 1: Maize Embryogenic Assays Using Agrobacterium-Mediated Transformation and Constitutively Expressed Morphogenic Genes

Vector Design

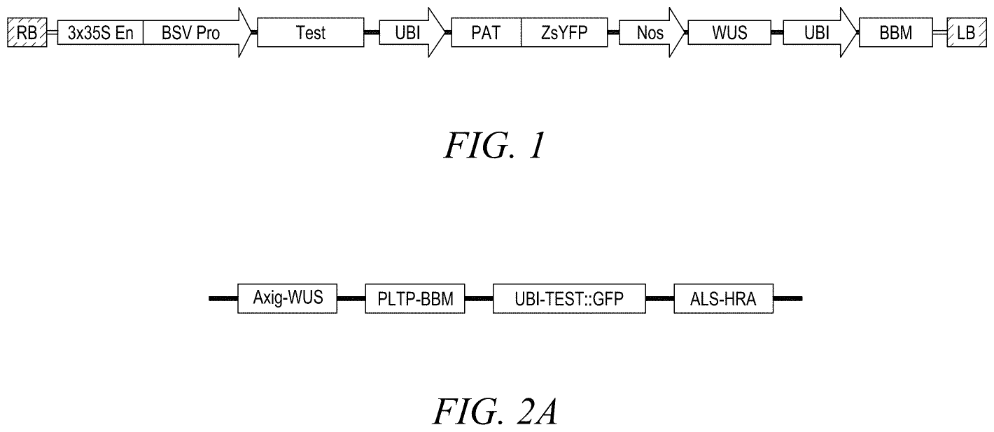

Sets of vectors were designed to compare the impacts on plant health attributable to the presence of one or more agronomically important genes of interest compared to neutral control genes. In this example, effects of test genes are detected at an early stage during maize transformation. FIG. 1 shows a representative vector design used in the experiments. The main feature of the vector system is the linkage of expression of a Test Gene (gene of interest or neutral control gene) with that of a combined selectable marker/visual marker to monitor transformed tissue growth and proliferation in real time. The vector also includes expression cassettes for morphogenic genes, such as BABYBOOM (BBM) and WUSCHEL (WUS) under control of constitutive Nos and Ubiquitin promoters, which enable high rates of transformation and growth of embryonic tissue.

Preparation Of Agrobacterium Suspension

Agrobacterium tumefaciens, harboring a binary vector, was streaked out from a −80° frozen aliquot onto solid LB medium containing 100 mg/L spectinomycin or appropriate selection agent and cultured at 28° C. in the dark for 2-3 days. A single colony (or multiple colonies) was picked from the master plate and streaked onto a plate containing 810D medium (5 g/l yeast extract, 10 g/l peptone, 5 g/l NaCl, adjust pH TO 6.8 with NaOH, 15 g/l bacto-agar, autoclave and cool to 60° C., then add 1 ml/l of 50 mg/ml spectinomycin) and incubated at 28° C. in the dark for 1-2 days. Agrobacterium cells were collected from the solid medium using 5 mL 700B medium (Agrobacterium infection medium, 4.3 g/l Murashige and Skoog (MS) basal salt mixture, 0.1 g/l myo-inositol, 1 g/l vitamin assay casamino acids, 68.5 g/l sucrose, 36 g/l glucose, 0.5 ml/l of 1 mg/ml nicotinic acid, 0.5 ml/l of 1 mg/ml pyridoxine-HCl, 2.5 ml/l of 0.4 mg/ml thiamine-HCl, 3 ml/l of 0.5 mg/ml 2,4-D, adjust pH to 5.2 with KOH, filter with 0.2 micron filter) with 1 ml/l of 100 mM acetosyringone (AS). The optical density of the suspension was adjusted to 0.35 at 550 nm using the same medium. The final Agrobacterium suspension was aliquoted into 2 mL micro-centrifuge tubes, each containing 1.5 mL of the 700B medium+AS suspension.

Maize Transformation

Maize inbred HC69 ears were chosen for quality of pollination, embryo color (milky) and embryo size. Other genotypes may be used. The optimal size of the embryos is 1.6-1.9 mm for most genotypes, however for some genotypes the optimal embryo size may be between 2.0-2.5 mm. Ears were surface-sterilized for 15-20 min in 20% (v/v) bleach (5.25% sodium hypochlorite) plus 1 drop of Tween 20 followed by 3 washes in sterile water. Immature embryos (IEs) were isolated from ears, placed in 1.5 ml of the 700B media with 1 ml/l of 100 mM AS and suspended for 20 minutes. Approximately 20 IEs from each donor ear were split evenly between each test vector to reduce any ear-specific bias in transformation. The solution was drawn off and the IEs were infected with 1.5 ml of Agrobacterium suspension. The tube was vortexed at a speed of 4-5 for 5-10 sec and incubated for 5 min. The suspension of Agrobacterium and IEs was poured onto 710B co-cultivation medium (4.3 g/l MS basal salt mixture, 0.1 g/l myo-inositol, 0.7 g/l L-proline, 0.5 g/l MES buffer, 20 g/l sucrose, 10 g/l glucose, 0.5 ml/l of 1 mg/ml nicotinic acid, 0.5 ml/l of 1 mg/ml pyridoxine-HCl (′7p), 10 ml/l of 0.1 mg/ml thiamine-HCl, 4 ml/l of 0.5 mg/ml 2,4-D, adjust pH to 5.8 with KOH, autoclave, cool to 60° C. and add of 0.1 ml/l 1M AS and 1 ml/l 10 mg/ml ascorbic acid). Any embryos left in the tube were transferred to the plate using a sterile spatula. The Agrobacterium suspension was drawn off and the IEs were placed axis side down on the media. The plate was sealed with Parafilm tape and incubated in the dark at 21° C. for 3 days of co-cultivation. The IEs were then transferred axis side down to RWC resting medium (PIBC2(20S10G)-Ag (4.3 g/l MS basal salt mixture, 2.39 g/l N6 macronutrients 10× (38F), 1.68 g/l potassium nitrate, 0.6 ml/l B5H MINOR SALTS 1000× (66A), 6 ml/l NaFe EDTA FOR B5H 100× (66B), 0.4 ml/l ERIKSSON'S VITAMINS 1000× (13009BA5E), 6 ml/l S&H VITAMIN STOCK 100× (45BASE), 2 ml/l thiamine-HCL at lmg/ml, 1.98 g/l L-proline, 0.3 g/l casein hydrolysate, 20 g/l sucrose, 10 g/l glucose, 1.6 ml/l 2,4-D 0.5 mg/ml (No. 2A), 0.49 ml/l of 0.1 M CuSO₄, 0.1 ml/l of 1 mg/ml BAP, 0.6 ml/l of 2 mg/ml dicamba, adjust pH to 5.8 with KOH, 3.5 g/l phytagel, autoclave, cool to 60° C.) and add 1 ml/l of 100 mg/ml cefotaxime (PhytoTechnology Lab., Shawnee Mission, Kans.) to control AGL-1 growth. The IEs were incubated at 26±2° C. under dim light for 4 days. YFP/CFP/GFP expression in embryos was monitored over time by visual capture of fluorescence (using appropriate excitation/emission filter sets). For data capture the 10 most representative embryos per treatment were chosen. The coleoptiles were removed from the IEs and transferred to DBC3(M1G) GT (green tissue) induction medium (4.3 g/l MS basal salt mixture, 0.25 g/l myo-inositol, 1.0 g/l casein hydrolysate, 30 g/l maltose, 0.69 g/l L-proline, 0.5 ml/l of 1 mg/ml BAP, 1 g/l glucose, 10 ml/l of 0.1 mg/ml thiamine-HCl, 2 ml/l of 0.5 mg/ml 2,4-D, 0.049 ml of 0.1M cupric sulfate adjust pH to 5.8 with KOH, 3.5 g/l phytagel, autoclave, cool to 60° C. and add 0.6 ml/l of 5 mg/ml bialaphos) supplemented with 100 mg/L cefotaxime and 3 mg/L bialaphos (for constructs containing the moPAT gene disclosed in U.S. Pat. No. 6,096,947, incorporated herein by reference in its entirety, as a selectable marker) for the first round of selection and incubated at 26±2° C. under dim light for 2 weeks. At 21 days post infection (d.p.i.), the tissue was subcultured on DBC3(M1G) GT induction medium containing 100 mg/L cefotaxime and 5 mg/L bialaphos. Expression was monitored every day for 2 weeks. Photo images were captured with the Leica Analysis Suite (Leica Microsystems Inc., 1700 Leider Lane, Buffalo Grove, Ill. 60089). Transformation frequency (total number of YFP sector expressing embryos/total number of embryos) as well as relative YFP-expressing sector area (total area of YFP-expressing stable sectors/total surface area of embryos) was recorded at 21 days and 28 d.p.i. (data not shown).

Rapid Embryogenic Callus Assay Using Fluorescent Protein ZsYellow (YFP) as a Visual Marker

A set of vectors was designed to determine if impacts on plant health attributable to the presence of one or more agronomically important genes of interest could be detected at an early stage during maize transformation. FIG. 1 shows a representative vector design used in the experiments. The main feature of the vector system is the linkage of expression of a Test Gene (gene of interest or neutral control gene) with that of a combined selectable marker/visual marker to monitor transformed tissue growth and proliferation in real time. The vector also included expression cassettes for morphogenic genes, such as BABYBOOM (BBM) and WUSCHEL (WUS), which enabled high rates of transformation, tissue growth and regeneration. The combined selectable marker/visual marker was phosphinothricin acetyltransferase (PAT) gene fused in frame to a fluorescent protein visual marker gene such as ZsYellow (YFP). The moPAT:YFP fusion gene cassette was driven by the strong maize ubiquitin promoter for optimal selection and robust fluorescent protein expression. Finally, the Test Gene (gene of interest or neutral control gene) in all cases was driven by an enhanced (3×35S enhancer repeats) version of the banana streak virus BSV promoter to ensure maximal expression and easier detection of any growth response. Typical experiments with a control vector expressing a neutral control gene (Test Gene) PMI (phosphomannose isomerase) gave on average about 30-100 independent events per immature embryo explant within 7-14 d.p.i. with Agrobacterium (data not shown). In contrast, vectors directing expression of genes negatively impacting plant health genes gave significantly fewer transformation events of lower intensity (data not shown).

Using this vector design and rapid transformation system two insecticidal proteins were tested, Gene A and Gene B, which each exhibited a low transformation frequency and poor cell growth when compared to controls in prior standard plant transformation experiments. In a first experiment Gene A expression was compared to the neutral control gene PMI expression in maize inbred HC69. A significantly higher number of healthy transformation events were obtained per immature embryo for vectors with the neutral control gene (PMI) compared to Gene A, 14 d.p.i. with Agrobacterium (data not shown).

All infiltrated embryos with the neutral control PMI gene were covered with new, YFP-positive, micro-embryo events. The overall fluorescence signal was very bright from multiple events per embryo, each expressing high levels of YFP. The Gene A infiltrated embryos on the other hand, showed less than ten such YFP-expressing events per infiltrated embryo. The overall YFP signal of Gene A infiltrated embryos was reduced over 20-fold in comparison to the PMI infiltrated embryos. This is consistent with the extreme cellular stress induced by Gene A production that was observed in multiple assay systems.

In a second experiment, the expression in maize of Gene B and PMI were compared over time post transformation and the relative fluorescent protein accumulation, which correlates to cell growth response, was quantified. Differential expression of YFP in Gene B (gene of interest) and PMI (neutral control gene) transformed cells (differential cell growth response) was seen as early as 3 d.p.i. and continued to differentiate dramatically (˜a 10- to a 20-fold differential) and was seen at 7, 10, and 14 d.p.i. (data not shown).

Rapid Embryogenic Callus Assay Using Fluorescent Protein CYAN (CFP) as a Visual Marker

To demonstrate that the assay system was not limited to the use of YFP as a visual monitor, an alternative fluorescent protein, Cyan Fluorescent Protein (CFP), was tested. In addition, other genes of interest were tested.

CFP gene was used to substitute for YFP in the cassette design and the moPAT::fluorescent protein fusion format was maintained. Except for reading blue fluorescence instead of yellow, the performance of this vector was the same. Test Genes PMI (neutral control gene), Gene B (gene of interest), Gene C (gene of interest), Gene D (gene of interest), and Gene E (gene of interest) were introduced into a vector containing a moPAT::CFP fluorescent protein fusion and tested in the maize callus growth assay as described above. CFP expression was monitored at 10-14 days post Agrobacterium infection (d.p.i.). The difference in CFP expression was very clear amongst the Test Genes at 10 d.p.i. and became more evident with time (data not shown). The results showed Gene E had the most impact on cell growth response followed by Gene D and then Gene B. By contrast, Gene C caused the least impact on cell growth response as evidenced by the most transgenic sectors and the brightest fluorescence (data not shown).

Rapid Embryogenic Callus Assay can be Applied to Alternative Tissue Types and Inbreds

The assay system described herein can be used with other maize inbreds as well as other plant tissues or cells, such as leaf tissue. When inbreds PH12BN (see U.S. Pat. No. 7,820,895) and PH184C (see U.S. Pat. No. 8,445,763) were transformed with Gene B (gene of interest) and PMI (neutral control gene) similar results, as those described above were obtained. PH12BN immature embryos (IEs) transformed with PMI (neutral control gene) and Gene B (gene of interest) showed a clear differentiation in event frequency and fluorescent protein intensity (data not shown). PH184C IEs transformed with PMI (neutral control gene) and Gene B (gene of interest) did not show such a clear differentiation in growth response by 10 d.p.i. however there was a clear differentiation in growth response by 14 d.p.i. (data not shown). The rapid embryogenic callus assay described herein also works when using alternative explants and was tested in PH184C leaf tissue. Leaf tissue transformed with PMI (neutral control gene) and Gene B (gene of interest) showed a clear differentiation in growth response (data not shown).

### Example 2: Maize Embryogenic Assays Using Agrobacterium-Mediated Transformation with Temporal and Spatial Promoter-Driven Morphogenic Genes

Rapid Maize Transformation Plant Response Assay System

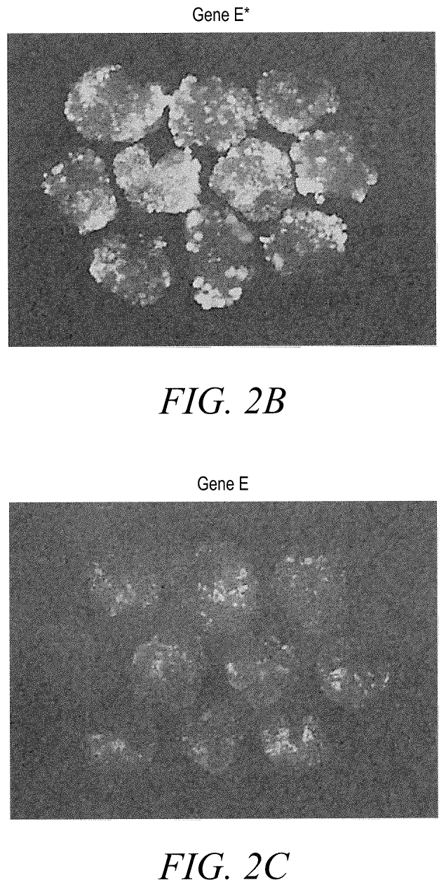

To improve the assay system described in EXAMPLE 1, methods of rapid transgenic plant recovery were incorporated to further evaluate growth response parameters and to evaluate the functionality of the Test Genes (genes of interest and neutral control genes). The vector design of EXAMPLE 1 was modified. The vector included the same morphogenic genes as in EXAMPLE 1 except with temporal/spatial specific promoters (FIG. 2A) that allow growth of maize tissue beyond the embryogenic phase and into whole plants in a very rapid manner (see US20170121722 incorporated herein by reference in its entirety). In addition, this new vector design uses the visual marker green fluorescent protein (GFP) fused in-frame to the Test Gene (gene of interest or neutral control gene) to allow real time quantification of transformed tissue growth and Test Gene expression between events. Finally, the high resistance allele (HRA) selectable marker for use with sulfonylurea and imidazoline herbicides was used in place of the moPAT selectable marker.

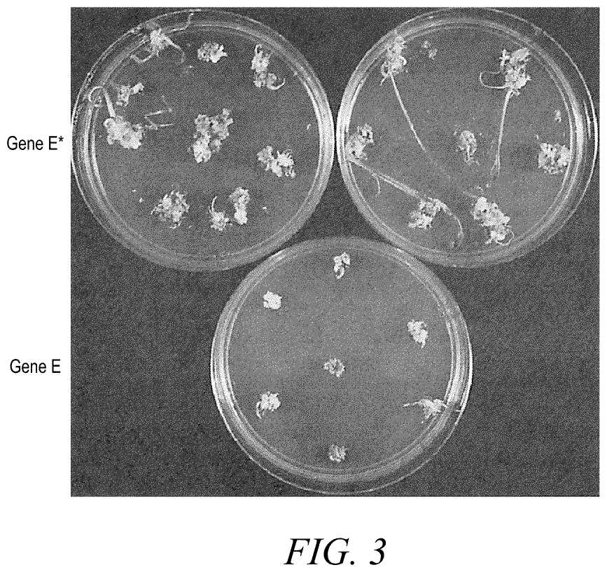

The utility of this test system was exemplified by introducing the Gene E gene of interest used in EXAMPLE 1, which had a significant impact on cell growth response, and comparing it to an isogenic control gene of interest with a single base substitution (Gene E*). This single base substitution results in expression of a non-inhibitory version of the Gene E insecticidal protein. The results show clear differentiation in growth response (numbers of transformed foci as well as transformed tissue proliferation) between the expression of Gene E (FIG. 2C) and its mutant version Gene E* (FIG. 2B). As shown in FIG. 2B, the presence of white in the Gene E* pictograph panel correlates to cell growth response (a greater number of transformed foci as well as transformed tissue proliferation) and the absence of white in the FIG. 2C Gene E pictograph panel correlates to less cell growth response (fewer transformed foci as well as less transformed tissue proliferation). Similar results were observed in the maize callus assay in EXAMPLE 1 when the expression of Gene E (gene of interest) was compared to the expression of PMI (neutral control gene). However, in this Rapid Maize Transformation assay the Test Gene (gene of interest or neutral control gene) was directly linked as a translational fusion to GFP (the N-terminal end being Gene E or Gene E* and C-terminal end being GFP) and thus transformed tissues that fluoresced were highly likely to be expressing the Test Gene (gene of interest or neutral control gene). In the maize callus assay described in EXAMPLE 1, transformed fluorescent tissues could have little to no Test Gene expression since the fluorescent marker gene and Test Gene (gene of interest or neutral control gene) were driven from separate promoter cassettes (FIG. 1). As shown in FIG. 3, this Rapid Maize Transformation assay allows for comparison of expression of the Test Gene (gene of interest or neutral control gene) to be carried out in transformed plants, which permits rapid determination of subtle performance issues between different genes/constructs not detectable at the earlier stages of tissue development.

Preparation of Agrobacterium Suspension

The same Preparation of Agrobacterium Suspension as in EXAMPLE 1 was performed.

Rapid Maize Transformation

Maize HC69 inbred ears were chosen for quality of pollination, embryo color (milky) and embryo size. The optimal size of the embryos is 1.6-1.9 mm for most genotypes, however for some genotypes the optimal embryo size may be between 2.0-2.5 mm. Ears were surface-sterilized for 15-20 min in 20% (v/v) bleach (5.25% sodium hypochlorite) plus 1 drop of Tween 20 followed by 3 washes in sterile water. Immature embryos (IEs) were isolated from ears and placed in 1.5 ml of the 700B medium with AS solution and suspended for 20 min. Approximately 20 IEs from each donor ear were split evenly between each test vector to reduce any ear-specific bias in transformation. The solution was drawn off and the IEs were infected with 1.5 ml of Agrobacterium suspension. The tube was vortexed at a speed of 4-5 for 5-10 sec and suspended in bacteria for 5 min. The suspension of Agrobacterium and IEs was poured onto 710B co-cultivation medium. Any embryos left in the tube were transferred to the plate using a sterile spatula. The Agrobacterium suspension was drawn off and the IEs were placed axis side down on the media. The plates were incubated in the dark at 21° C. for 1 day of co-cultivation. The IEs were transferred axis side down to resting medium (PIBC3(10M10S5G)-Ag (4.3 g/l MS basal salt mixture, 2.39 g/l N6 macronutrients 10× (38F), 1.68 g/l potassium nitrate, 0.6 ml/l B5H MINOR SALTS 1000× (66A), 6 ml/1 NaFe EDTA FOR B5H 100× (66B), 0.4 ml/l ERIKSSON'S VITAMINS 1000× (13009BA5E), 6 ml/l S&H VITAMIN STOCK 100× (45BASE), 2 ml/l thiamine-HCL at lmg/ml, 1.98 g/l L-proline, 0.3 g/l casein hydrolysate, 10 g/l sucrose, 10 g/l maltose, 5 g/l glucose, 1.6 ml/l 2,4-D 0.5 mg/ml (No. 2A), 0.049 ml/l of 0.1 M CuSO₄, 0.5 ml/l of 1 mg/ml BAP, 0.6 ml/l of 2 mg/ml dicamba, adjust pH TO 5.8 with KOH, 3.5 g/l phytagel, autoclave, cool to 60° C.) and add 1 ml/l of 100 mg/ml cefotaxime (PhytoTechnology Lab., Shawnee Mission, Kans.) to control AGL-1 growth. The IEs were incubated at 26+2° C. in dark for 7 days. Fluorescent protein expression in embryos was monitored over time (7, 10, and 14 d.p.i) by visual capture of fluorescence (using appropriate excitation/emission filter sets). For data capture the 10 most representative embryos per treatment were chosen. The coleoptiles were removed from the IEs and transferred to 289Q medium (4.43 g/l MS basal salt mixture with vitamins (M519), 0.1 g/l myo-inositol, 1.25 ml/l of 1 mg/ml cupric sulfate, 0.7 g/l L-proline, 60 g/l sucrose, adjust pH to 5.6 with KOH, autoclave, cool to 60° C. and add sterile 0.5 ml/l of 2 mg/ml IAA, 1 ml/l 0.1 mM ABA, 0.1 ml/l 100 mg/ml thidiazuron, 0.5 ml/l 1 mg/ml zeatin) containing 150 mg/L cefotaxime and 0.5 mg/L imazapyr for the first round of selection and incubated at 26+2° C. in dark for 2 weeks. At 22 days post-infection, the tissue was subcultured on 13158H medium (4.43 g/l MS basal salt mixture with vitamins (M519), 40 g/l sucrose, adjust pH to 5.6 with KOH), autoclave, cool to 60° C. and add sterile 150 mg/L cefotaxime and 0.5 mg/L imazapyr) and placed in low light. Transformation frequency (total number of GFP sector expressing embryos/total number of embryos) was recorded at 22 days and at 28 days post infection.

### Example 3: Maize Embryogenic Assays Using Agrobacterium-Mediated Transformation with Cre-Mediated Excision of Temporal and Spatial Promoter-Driven Morphogenic Genes

The methods described in EXAMPLES 1 and 2 above were further improved to enhance the production of mature maize plants by incorporating a Cre/Lox site-specific recombinase system into the vectors to mediate excision of the morphogenic genes after the initial transformation period (US Patent Publication 2017/0121722, incorporated herein by reference in its entirety). An intron-disrupted version of the Cre recombinase was placed under transcriptional control of the heat-shock inducible ZM-HSP26 promoter (US Patent Publication 2017/0121722, incorporated herein by reference in its entirety). The effects of test vs. neutral-control genes was observed and quantified throughout the plant growth cycle. it was noted that different families of insecticidal proteins differ in their temporal effects on plant development. In some cases, dramatic effects were seen at the initial transformation stage as having reduced transformation frequency and intensity of test gene-GFP fusion expression. In other examples, normal rates of initial transformation and GFP-fusion expression were observed, but the plants died or were growth stunted at various stages of maturity.

### Example 4: Plant Response Assay in Bean Leaves Via Agrobacterium-Mediated Transient Expression

Transient expression of a Test Gene (gene of interest or neutral control gene) in plant leaf tissue was initiated via infiltration of a suspension of Agrobacterium harboring a T-DNA expression vector containing the Test Gene. Peak transient expression of a Test Gene in leaf tissues typically occurred three d.p.i. If a Test Gene (i.e. an insecticidal gene of interest) was known to have an impact on plant health and exhibited a visually discernable phenotype in the infected leaf area then this could be the basis of a rapid screening method for other Test Genes.

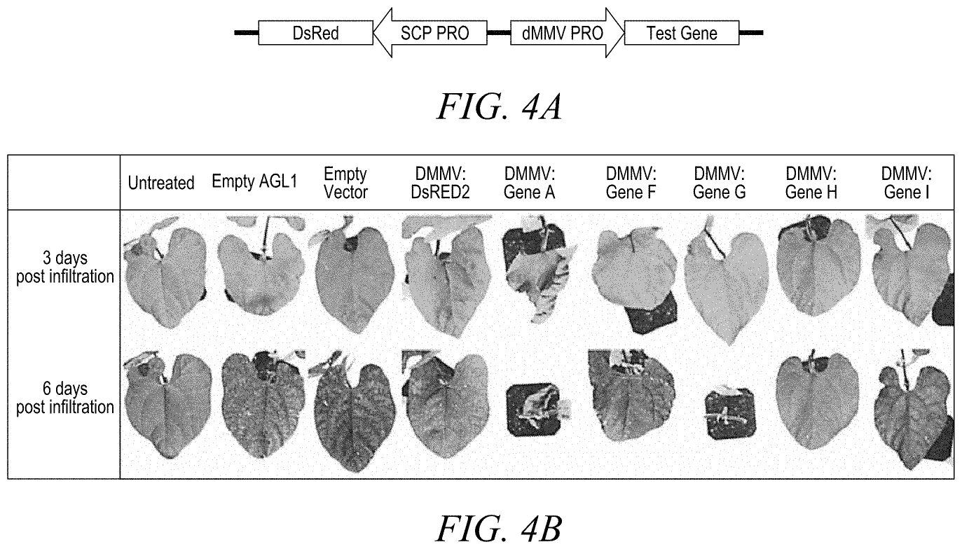

To test this concept, a T-DNA vector was developed as shown in FIG. 4A. Key features of the vector are an expression cassette using the very strong enhanced mirabilis mosaic virus promoter (DMMV) (U.S. Pat. No. 6,420,547 incorporated herein in its entirely) to drive expression of a Test Gene and in the opposite direction a synthetic constitutive promoter (SCP1) (U.S. Pat. No. 6,072,050 incorporated herein in its entirely) driving expression of a red fluorescent protein visual marker DsRed2 (Clontech). Vectors as shown in FIG. 4A were constructed for Test Genes DsRED2 (neutral control gene), Gene A (gene of interest), Gene F (gene of interest), Gene G (gene of interest), Gene H (gene of interest), and Gene I (gene of interest). The Test Genes were inserted downstream of the DMMV promoter and the vectors were introduced into Agrobacterium tumefaciens strain AGL1. The Agrobacterium cultures were resuspended in 10 mM MgSO4, 400 μM AS, and 1 mM dithiothreitol, normalized to an OD600 of 1.0 and force infiltrated into unifoliate stage leaves of bean (common bean; Phaseolus vulgaris spp.). Post infiltration the bean plants were placed in a growth chamber at 26° C. for 3 or more days and then removed for image capture phenotype documentation. In repeated tests, the expression of the Gene A and Gene G genes led to tissue necrosis within 2-7 days post infiltration whereas expression of DsRED2, Gene F, Gene H and Gene I genes gave a response similar to infiltration with an empty A. tumefaciens strain AGL1 (FIG. 4B).

### Example 5. Using Yeast as a High-Throughput Plant Response Surrogate Assay

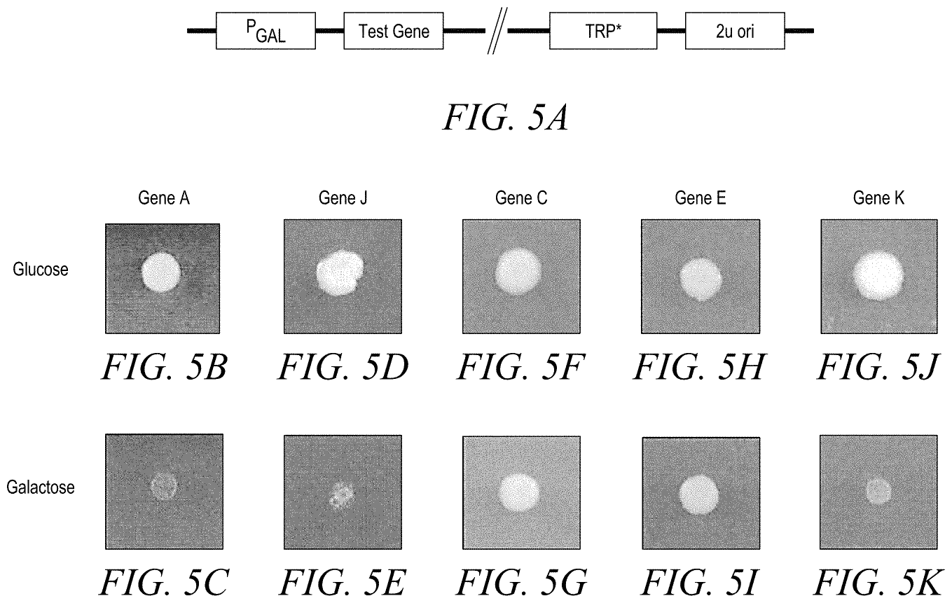

Over production of certain proteins in plants may impact plant health. Several Test Genes (Gene A, Gene J, Gene C, Gene E, Gene K) known to have a range of impacts on plant health from none to severe were introduced into pESC-TRP (Agilent) downstream of the GAL1 promoter by in vivo homology based cloning in S. cerevisiae host strain YPH500 (Agilent) (FIG. 5A). A BamH1/Sal1 cut vector fragment was mixed with PCR amplified Test Gene fragments having approximately 40 bp of homology on each end to the vector DNA sequence and YPH500 competent cells and plated onto synthetic agar medium complete with all nutrients except tryptophan and having glucose as the carbon source (CM glucose agar plates minus Tryptophan (Trp-) Teknova Cat #C3555) to allow for tryptophan prototrophy selection. Positive clones identified by PCR/sequencing were re-arrayed in 96-well format and replica plated onto synthetic agar medium complete with all nutrients except tryptophan and with either glucose or galactose as the carbon source. Assay plates were placed at 30° C. then photographed after 48 and 72 hours of incubation.

Colony size on glucose vs. galactose for each Test Gene was then compared. Test Gene Gene C (FIG. 5F and FIG. 5G) had a neutral impact on growth response in all plant assays and had a neutral impact on growth response in this yeast assay. Test Genes Gene A (FIG. 5B and FIG. 5C) and Gene J (FIG. 5D and FIG. 5E) both caused a negative growth response in this assay. Gene E (FIG. 5H and FIG. 5I) did not cause a negative growth response unless only the C-terminal end (Gene K) was expressed (FIG. 5J and FIG. 5K). This gene fragment was also shown to have a negative growth response in the bush bean assay (data not shown).

### Example 6. Protoplast Assay

Viable plant protoplasts were generated from enzymatic digestion of freshly harvested plant leaf tissue as described below for maize. Endotoxin-free DNA was prepared for expression vectors for the Test Genes (Gene A (gene of interest), Gene L (gene of interest), and PMI (neutral control gene). Defined amounts of vectors containing the genes of interest and the neutral control gene were transfected into the protoplasts using a standard PEG method. The protoplasts also received equal amounts of a vector containing a luciferase reporter gene. The protoplasts were incubated overnight to allow gene expression. After incubation for 16-18 hours, a luciferin substrate was added to the transfected protoplasts and luciferase enzyme activity was measured. The luciferase counts were read in live cells. Luciferase activity was directly proportional to ATP levels in the protoplasts, and higher luciferase counts correspond to healthier protoplasts. In this way, the relative impact on plant health of a Test Gene was determined. The impact on plant health (plant cell growth) was presented as percent increase or reduction in luciferase activity from that of the neutral control gene.

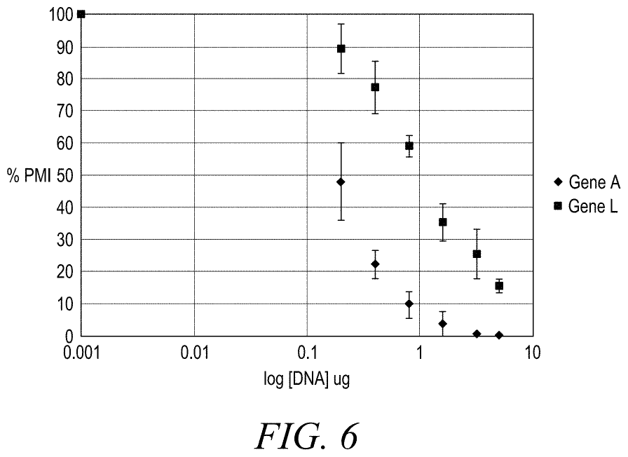

FIG. 6 shows that Gene L (squares) had approximately a 20-fold less impact on plant health than Gene A (diamonds). Protoplasts cells were transfected with increasing concentrations of Test Gene encoding DNA vectors. Increasing amounts of DNAs in the transfection showed a reduction in the luminescence signals from both vectors (Gene A and Gene L), due to higher transient protein production and increased impact on plant health as quantified by reduced luciferase activity. The luminescence was normalized as percentage of the expression of PMI (neutral control gene). The experiments were repeated three times, each in triplicate and averaged.

Preparation of Protoplasts from Maize Leaf Tissue

Six to ten-day old light grown HC69 maize seedlings were cut midway up the stalk. The leaves were stored in autoclaved ddH₂O until use. Leaves were washed and dried gently, stacked and folded one time and then sliced into very fine strips (<1 mm) using a fresh razor blade. Approximately 10 mL of enzyme solution was poured into a 60 mm Petri-dish and then the sliced tissue was transferred into the dish. The tissue was mixed briefly in the enzyme solution. The Petri-dish was placed, without its lid, into a vacuum chamber. The chamber was sealed and the vacuum was turned on for 20 minutes. The Petri-dish was transferred to a shaking platform and set to approximately 40 rpm (speed 2.2). The Petri-dish was covered to block light and shook for 75 minutes. 75 μm nylon mesh was washed with water then W5 solution. The enzyme solution was shaken at approximately 80 rpm (speed 3) for 1 minute to release the protoplasts. The protoplasts were filtered through the nylon filter into a 50 mL conical tube. The undigested tissue was discarded. 0.25 volumes of 0.2M CaCl₂) was added to the protoplasts and mixed by inverting. The protoplasts were spun in a swinging bucket centrifuge (3 minutes at 160×g at 18° C.) to pellet the protoplasts. The supernatant was aspirated away from the pellet and the pellet was resuspended in 15 mL W5 solution. The protoplasts were spun again to pellet and resuspended in 15 mL W5 solution and incubated on ice for 30 minutes (or longer). The protoplasts were counted, spun again to pellet and then resuspended in MMG solution.

Transfection of Protoplasts

DNA was added to wells of a 48 well block (Axygen, Cat. #P-5ML-48-C-S) 5 μg of luciferase reporter gene in a vector behind a strong constitutive promoter and 5 μg of Test Gene in the same vector per well. The block was spun briefly to collect all DNA on the bottom of each well. 100 μl of protoplasts (approximately 4×10⁵ protoplasts/ml in MMG solution) was added to each well and the block was tapped to mix. 100 μl of 40% PEG solution was added per well and the block was tapped to mix. Protoplasts+PEG were incubated for 20 minutes at room temperature. 400 μl of WI solution was added per well and the block was tapped to mix. Another 400 μl of WI solution was added per well to bring the final volume up to 1 ml. The block was tapped to mix. The wells were sealed with AirPore film (Qiagen, Inc.) and incubated in the dark at room temperature for 16-24 hours.

Luciferase Assay

Protoplasts were pelleted in a 48 well block by centrifugation (3 minutes at 160×g at) 18°. Protoplasts were aspirated and resuspended in 100 μl WI per well and mixed by tapping the block. 50 μl of protoplasts were transferred to wells of round bottomed white plates. 50 μl of Potassium D-Luciferin (Gold Biotech., Cat #LUCK-1G) was added to each well at 10 mg/ml in WI and mixed gently, then incubated for 5 min. in the dark at room temperature. Luciferase signal was read on Berthold Tech Mithras plate reader (Berthold Technologies GmbH & Co. KG, Calmbacher Str. 22. 75323 Bad Wildbad, Germany).

Solutions

Enzyme Solution: 0.6% (wt/v) Cellulase, 0.1% (wt/v) Pectolyase, 0.1% (wt/v) BSA, 400 μM Sorbitol, 1 mM CaCl₂), 10 mM KCl, 5 mM MES

W5 Solution: 154 mM NaCl, 125 mM CaCl₂), 5 mM KCl, 0.1% (wt/v) PVPP

MMG Solution: 0.4M Mannitol, 15 mM MgCl₂, 4 mM MES, 5 mM Glucose

WI Solution: 0.5M Mannitol, 0.1M Ca(NO₃)₂, 4 mM MES, 20 mM KCl, pH=5.7

40% PEG: 0.4M Mannitol, 40% PEG4000, pH=10.0

### Example 7. Callus Assay

Callus tissue from an immature zygotic embryo was used to reveal unexpected poor plant phenotypes by tracking the rate of growth of callus tissue expressing a construct containing a gene of interest compared to the rate of growth of callus tissue expressing a construct containing a benign gene. The tissue generated with a benign gene was considered control tissue for the comparison. A benign gene construct was designed to express only a selectable marker(s) required to transform the species of interest to generate callus. A slower growth rate of tissue expressing a gene of interest vs. the growth rate of control tissue was considered an indication that the gene of interest produced a poor plant response in transformed plants. Maize transformation was performed as described in Ishada, Y., et al., (1996) Nat. Biotechnol. 14: 745-750. No morphogenic genes were utilized in this assay system, so an extended phase of transformed callus tissue was observed and differences between test genes was quantified.

Additionally, growth rate comparisons between callus tissue expressing a gene of interest vs. a benign gene were made under growth conditions designed to subject the tissue to stress. This approach was used to uncover plant responses not seen under normal growth conditions. To determine this, the callus tissue was divided into an equal number of relatively even sized portions, then one half of each subdivided set of tissue was allowed to grow under stress conditions involving high osmoticum, and/or heat, and/or light while the other half was grown under normal conditions. In addition to tracking growth rate changes between the different culture conditions and compared to the growth rate of control tissue under those culture conditions, other features of the callus tissue were measured and revealed an unexpected poor response to the accumulation of the protein under study. An example of this type of phenotype was a color change of the tissue which resulted from accumulation of pigments such as anthocyanins and xanthophylls. The accumulation of purple and/or reddish pigments, especially anthocyanins, was associated with plant stress responses. The increased accumulation of such pigments under normal and/or stress culture conditions was considered a marker of poor plant response.

### Example 8. Osmotic Stress Test

Maize Embryo and Event Isolation

Callus events were created from callus-forming embryos. Hi II (Armstrong, C. L., Green, C. E. and Phillips, R. L. (1991) Development and availability of germplasm with high type-II culture formation response. Maize Genet. Coop. Newsletter 65, 92-93) ears were surface sterilized with a 40% Clorox solution (400 mls bleach, 4 drops Tween 20 micro detergent, filled to 1000 mls total volume with de-ionized water) for 15 minutes, and rinsed 3 times with sterile water. 8 to 9 mm embryos were isolated and placed in 561Q medium (Murashugie & Skoog based liquid culture media) in a 2 ml screw cap tube until infection.

Agrobacterium was prepared by streaking AGL1 Agrobacterium with the selected construct onto minimal media plates, and placed upside down in the dark for 2 to 3 days at 28° C. One day prior to transformation, an overnight AGL1 Agrobacterium culture was started by placing a generous loop of AGL1 Agrobacterium into 3 mls of LB broth with appropriate selection (1800 mg/liter spectinomycin), and shaken at 250 rpm for 12 to 16 hours in dark at 28° C.

On the day of transformation, 561Q medium+100 μM AS in DMSO was created. The AGL1 Agrobacterium was pelleted and resuspended in 1000 μl 561Q medium+AS (infection solution). Final OD reading at 600 nm ranged from 0.20-0.29.

Once the embryos were isolated, and the Agrobacterium infection solution was created, the AGL1 Agrobacterium suspension was placed in an embryo isolation tube, the tube was capped and gently mixed for 15 minutes. The AGL1 Agrobacterium solution was removed from the embryo tube and replaced with 2 ml sterile media to wash the AGL1 Agrobacterium off the embryos. The embryos were resuspended and dumped onto a 562Q medium plate. The excess liquid was gently removed from around the embryos, and the embryos were allowed dry for approximately 2 to 4 hours. The embryos were adjusted so the adaxial side was upright, and the abaxial side was touching the media. The plates were placed in a 21° C. chamber in the dark for 3 to 4 days.

At the end of the co-culture period, the plates were transferred to a 28° C. for 3 to 4 days. Approximately one week after embryo isolation, the embryos were transferred to 13268A medium for selection and placed in dark at 28° C. Sub-culturing occurred every 2 weeks thereafter onto 13268A medium to prevent AGL1 Agrobacterium overgrowth. Events started to form after 3 to 4 weeks on selection. The number of events was counted, and the % transformation frequency was calculated. Once events started to form they were transferred to individual plates of 13268A medium for bulking up of callus. Individual events were subcultured every 2 weeks as appropriate.

Event Preparation for Osmotic Stress Test and Data Capture

Two weeks after events were placed on individual plates of 13268A medium for bulking up actively growing tissue, the events were placed on two types of media 13268A medium with no additional sorbitol and 13268A medium supplemented with 50 grams per liter sorbitol (275 μM sorbitol) (Osmotic Stress medium). One to two days before the first imaging, equal amounts of 0.5 to 1 cm² callus from the same growing area of the event were placed on the two test media in the same orientation.

Plates of the tissue were stored in the dark until the starting day of imaging, when they were photographed with the Leica Analysis Suite (Leica Microsystems Inc., 1700 Leider Lane, Buffalo Grove, Ill. 60089). After imaging (week 0), the 13268A, no sorbitol medium plates were placed in the dark culture room at 28° C. (RD conditions), and plates containing additional sorbitol were placed in the incubator under ˜300 μmole m-2 s-1, 33° C. (OST conditions). Plates were imaged again weekly at 1 and 2 weeks from placement on the 13268A, no sorbitol medium plates and the Osmotic Stress medium.

The 0, 1 and 2 week Jpeg images were opened in the Leica Analysis Suite, and callus area and % anthocyanin coloring was determined using the Leica Phase Expert function.

Gene a Osmotic Stress Test

Hyperspectral imaging was used to analyze plant health. Hyperspectral imaging is the collection of individual images from at least ten or more wavelengths. A spectrum is thus collected for each pixel. Wavelengths can range from the UV out into the short wave infrared and beyond. Imaging plant pixels across a spectrum can identify differences in composition such as pigments or water. A plant pixel devoid of chlorophyll but with high levels of anthocyanin will have very different spectra than a healthy green leaf pixel. Visually a plant with high levels of anthocyanin will appear red or brown. An image of the plant scene for each wavelength may be generated, which provides a mechanism for studying the spatial relationship in the plant between these compositional differences. For example, high levels of anthocyanin in the lower leaves of a maize plant are an indicator of stress. High dimensional imaging data cubes collected with a hyperspectral imager may be used to study photosynthesis, water and nitrogen stresses, the presence of disease and insects, and genetic sources of structural and compositional differences in plants.

In this experiment, callus area was measured at 0 weeks and 2 weeks, and the % of pixels that had accumulated red or green color during the 2 weeks of treatment was calculated.

A callus line (1) was generated with a trait cassette that expressed MoPAT only and served as a benign control. A callus line (2) was generated with a trait cassette that expressed MoPAT and Gene D and served as a positive control for a poor phenotype. A test callus line (3) was generated with a trait cassette that expressed Gene A, MoPAT, and PMI. Both callus lines (2) and (3) grew to a significantly smaller size after two weeks than callus line (1) even under regular growth conditions (RD). This difference was exacerbated under stress conditions. Callus area for callus line (1) in the OST assay increased an average of 2.97% over 2 weeks, while no growth was seen for callus line (2) and callus line (3) increased 0.45%. In addition, under stress, a typical marker of poor plant phenotype, anthocyanin, accumulated to high levels in both callus lines (2) and (3).

Monitoring the change in callus growth over time under non-stress and stress conditions compared to a benign control callus predicted stress responses seen in T0 and further generations. The generation of Gene A T0 plants over multiple experiments occurred at a much lower rate than expected (approx. 78% reduction in the event regeneration frequency) and those Gene A T0 plants that did survive expressed very low levels of Gene A and did not thrive to seed set.

Gene B Variants B1, B2, and B3 Osmotic Stress Test

Callus events were generated using trait cassettes expressing variants of Gene B (Gene B1, Gene B2 and Gene B3). These variants accumulated significantly more anthocyanin in their cells, as measured by % red pixels, after two weeks under stressed conditions than did tissue generated by transformation with a Control trait cassette containing only a selectable marker. The early observations in the callus assays, shown in Table 1 for Gene B variants (Gene B1, Gene B2 and Gene B3) correlated with the health of T0 corn plants generated with similar vectors in that on average only 78% of the plants generated were healthy at 5 weeks of age.

TABLE 1

| | | | average |
|---|---|---|---|
| | average % tissue area with | | expression at |
| | red pixels at 2 weeks | | beginning of |
| Trait | regular growth | OST | 2 wk assay |
| Cassette | conditions (RD) | conditions | (ppm) |
| Control | 0 | 15 | 0 |
| Gene B1 | 0 | 22 | 446 |
| Gene B2 | 0 | 20 | 84 |
| Gene B3 | 0 | 25 | 777 |

Media

561Q media comprises 4.0 g/L N6 basal salts (SIGMA C-1416), 1.0 mL/L Eriksson's Vitamin Mix (1000×SIGMA-1511), 0.5 mg/L thiamine HCl, 6.85% sucrose, 3.6% glucose, 1.5 mg/L 2,4-D and 0.69 g/L proline, with adjustment to pH 5.2 with KOH. Media was filter sterilized before use.

562Q media comprises 4.0 g/L N6 basal salts (SIGMA C-1416), 1.0 mL/L Eriksson's Vitamin Mix (1000×SIGMA-1511), 0.5 mg/L thiamine HCl, 3% sucrose, 2 mg/L 2,4-D and 0.69 g/L proline, with adjustment to pH 5.8 with KOH. Media is autoclaved, and 0.85 mg/L silver nitrate and 19.6 mg/L AS is added.

13268A media comprises 4.0 g/L N6 basal salts (SIGMA C-1416), 1.0 mL/L Eriksson's Vitamin Mix (1000×SIGMA-1511), 0.5 mg/L thiamine HCl, 3% sucrose, 1.5 mg/L 2,4-D, 0.69 g/L proline and 0.5 g/L MES, with adjustment to pH 5.8 with KOH. Media solidified with 0.8% agar, and 0.85 mg/L silver nitrate, 150 mg/L Timentin and 3 mg/L bialaphos is added post-autoclaving.

### Example 9: Arabidopsis thaliana

The rapid generation time, small size, ease of growth and ease of DNA transformation makes Arabidopsis thaliana a good model for crop plants. Reductions in transformation frequency, growth rate, plant mass and seed production with a test gene may be useful measures of a negative phenotype when compared to a benign control gene. Transformation using the floral dip method as described in Bent, 2006. Methods Mol. Biol. 343: 87-103 using a culture of Agrobacterium tumefaciens GV3101 containing a test gene or a neutral gene expression cassette each of which is designed to produce the respective proteins in the plant. The genes may be translationally coupled to a reporter such as GFP for rapid detection of relative expression rates and protein accumulation levels. Transformants encoding proteins causing a strong negative phenotype may fail to yield any stably transformed events or at a significantly reduced frequency compared to a benign control gene. Reductions in growth rate, plant mass and seed production with a test gene compared to a benign control gene can also be used to quantify a potential negative phenotype. Numerous imaging methods and algorithms can be used to further quantify the negative effects of test gene expression. Most benign proteins produce a strong fluorescent signal when fused to GFP, whilst proteins causing a negative plant response fail to accumulate GFP or GFP is accumulated at a significantly reduced rate. GFP fusion accumulation can be readily quantified by the fluorescent signal.

### Example 10: Soybean Hairy Root Assay

Three binary test vectors were used to test the impact on plant health of genes of interest Gene M and Gene N. Binary test vector A contained a bialaphos selectable marker expression cassette and an expression cassette containing a gene of interest, Gene M, translationally fused to GFP (Gene M::GFP) within the T-DNA borders. Binary test plasmid B contained a bialaphos selectable marker expression cassette and an expression cassette containing a gene of interest, Gene N, translationally fused to GFP (Gene N::GFP) within the T-DNA borders. Binary test vector C contained a bialaphos selectable marker expression cassette and a neutral fluorescent protein gene (GFP) expression cassette within the T-DNA borders. The use of fluorescent proteins, such as GFP or alternatively, DsRED, provided noninvasive detection of gene expression in living cells without the use of additional substrates. Real time visualization of gene expression was therefore observed. The plasmids (binary test plasmids A, B and C) were introduced into the Agrobacterium rhizogenes K599 strain by electroporation and cultured for 2-3 days on LB agar plates (10 g/L tryptone, 5 g/L yeast extract, 5 g/L NaCl, 8 g/L agar, see also, Sambrook J, Fritsch E F, Maniatis T (1989) Molecular cloning: a laboratory manual. Cold Spring Harbor Laboratory Press, New York) supplemented with 100 mg/L kanamycin at 28° C. to provide Kanamycin-resistant colonies. Kanamycin-resistant colonies were then grown to an OD of 1.0-1.5 at 600 nm in LB liquid medium supplemented with 100 mg/L kanamycin and frozen glycerol stock cultures were prepared and stored at −80° C. for future use.

Seeds [Glycine max (L.) Merr.] of soybean genotype 93Y21 were surface-sterilized by soaking in 20% (v/v) commercial bleach [5.25% (v/v) sodium hypochlorite] with Tween 20 (0.1%) for 20 min and then rinsed 6-7 times in sterile distilled water. Sterilized seeds were germinated on sucrose (0.5%) and agar (1.2%) medium under 16 h light (30-45 μE/m²/s, cool-white fluorescent lamps) at 25° C. Plant inoculation was conducted as follows: The day before transformation, a 5 ml culture of A. rhizogenes K599 with binary test vector A, or binary test vector B, or binary test vector C was grown in LB medium containing 100 mg/L kanamycin and then placed on a shaker incubator at 250 rpm overnight at 28° C. On the day of transformation, log phase A. rhizogenes K599 cells were centrifuged at 1,500×g for 10 minutes and cell pellets were diluted to an OD of 0.5 at 600 nm with liquid MSG co-cultivation medium [(Murashige and Skoog 1962) basal nutrient salts, B5 (Gamborg et al. 1968) vitamins and 1% sucrose (pH 5.2)] and used as the inoculum.

Cotyledons were harvested from either 4- to 5-day old seedlings or overnight imbibed seeds and were inoculated by uniformly wounding the abaxial and adaxial sides several times with a scalpel in an inoculum of A. rhizogenes K599 strain containing the binary test vector being tested. Then cotyledons were cultured abaxial side up on filter paper immersed in sterile distilled water and incubated under 16 h light (30-45 μE/m²/s, cool-white fluorescent lamps) at 25° C. Three days after inoculation, cotyledons were transferred to and cultured abaxial side up on solid MXB medium [MS (Murashige and Skoog 1962) basal nutrient salts, B5 (Gamborg et al. 1968) vitamins and 3% sucrose (pH 5.7)] with 3 g/L Gelrite (Greif Bros. Corp., East Coast Division, Spotswood, N.J., USA) in petri dishes (100 mm diameter, 25 mm deep). Timentin (300 mg/L) was added to inhibit the growth of A. rhizogenes and bialaphos (5 mg/L) was added to the MXB medium to select transformed hairy roots.

It was previously demonstrated in the bush bean assay that Gene M expression caused no phenotypic changes while expression of Gene N induced necrosis (data not shown). For the hairy root assay the two genes of interest (Gene M and Gene N) were fused to GFP, as described above, to track gene expression in real time in newly transformed tissues. The genes of interest were controlled by the strong constitutive Arabidopsis Ubiquitin 10 promoter and linked downstream to a Nos promoter driven BAR gene encoding resistance to the herbicide bialophos as the selectable marker. A. rhizogenes K599 harboring these test vectors was co-cultivated with soy cotyledon explant tissues and bialophos resistant/GFP+hairy root formation was monitored. After four weeks of root development results showed a high degree of proliferation and strong GFP fluorescence in hairy roots transformed with the GFP control (test vector C) and in hairy roots transformed with Gene M::GFP (test vector A). However, GFP fluorescence in hairy roots transformed with Gene N::GFP (test vector B) led to fewer and weaker bialophos resistant roots—none of which expressed any significant amount of GFP (data not shown). These results corroborated the relative impact on plant health observed from expression of these genes of interest in the bush bean assay (data not shown) and indicated that the hairy root assay also predicts the effects on plant health of genes of interest.

All patents, publications, and patent applications mentioned in the specification are indicative of the level of those skilled in the art to which this disclosure pertains. All patents, publications, and patent applications are herein incorporated by reference in their entirety to the same extent as if each individual patent, publication, or patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims.

1. A method of determining an impact on plant health of a gene of interest comprising:
a) providing a first plant cell and a second plant cell;
b) transforming the first plant cell with a first cassette comprising a gene of interest;
c) transforming the second plant cell with a second cassette comprising a neutral control gene;
d) culturing
i) the first transformed plant cell for expression of the gene of interest; and
ii) the second transformed plant cell for expression of the neutral control gene; and
e) determining the impact of expression of the gene of interest on plant health relative to expression of the neutral control gene.

2. The method of claim 1, wherein the first plant cell and the second plant cell is selected from the group of an alfalfa plant, an Arabidopsis plant, a barley plant, a broad bean plant, a broccoli plant, a bush bean plant, a cabbage plant, a canola plant, a cassava plant, a cauliflower plant, a clover plant, a cotton plant, a kale plant, a maize plant, a millet plant, a mustard plant, an oat plant, a pea plant, a rice plant, a rye plant, a safflower plant, a Setaria plant, a sorghum plant, a soybean plant, a sugarcane plant, a sunflower plant, a switchgrass plant, a tobacco plant, a tomato plant, a triticale plant, a turf grass plant, and a wheat plant.

3. The method of claim 1, wherein the first plant cell and the second plant cell is from the same plant.

4. The method of claim 3, wherein the first plant cell and the second plant cell of the maize plant is an immature embryo.

5. The method of claim 3, wherein the first plant cell and the second plant cell of the bush bean plant is a leaf.

6. The method of claim 3, wherein the first plant cell and the second plant cell of the soybean plant is a leaf.

7. The method of claim 3, wherein the first plant cell and the second plant cell of the soybean plant is an immature cotyledon.

8. The method of claim 3, wherein the first plant cell and the second plant cell of the soybean plant is an imbibed mature cotyledon.

9. The method of claim 3, wherein the first plant cell and the second plant cell of the soybean plant is an embryonic axis.

10. The method of claim 3, wherein the gene of interest is selected from the group of a gene conferring pest resistance, herbicide resistance, stress tolerance, drought resistance, nitrogen use efficiency (NUE), disease resistance, and an ability to alter a metabolic pathway.

11. The method of claim 10, wherein the neutral control gene is selected from the group of a chloramphenicol acetyl transferase (CAT) gene, a fluorescent protein (FP) gene, a phosphomannose isomerase (PMI) gene, a β-glucuronidase (GUS) gene, and a housekeeping gene.

12. The method of claim 11, wherein the first cassette further comprises a promoter operably linked to the gene of interest for expression of the gene of interest in the first plant cell and the second cassette further comprises a promoter operably linked to the neutral control gene for expression of the neutral control gene in the second plant cell.

13. The method of claim 12, wherein the promoter of the first cassette and the promoter of the second cassette is the same promoter.

14. The method of claim 13, wherein determining the impact of expression of the gene of interest on plant health relative to expression of the neutral control gene is a visual observation of a plant tissue.

15. The method of claim 14, wherein the visual observation is selected from the group of anthocyanin pigment production of the plant tissue, browning of the plant tissue, necrosis of the plant tissue, and growth of the plant tissue.

16-124. (canceled)

125. A method of determining an impact on plant health of a gene of interest comprising:
e) providing a first yeast cell and a second yeast cell;
f) transforming the first yeast cell with a first cassette comprising a gene of interest;
g) transforming the second plant cell with a second cassette comprising a neutral control gene or no gene;
h) culturing
iii) the first transformed yeast cell for expression of the gene of interest; and
iv) the second transformed yeast cell for expression of the neutral control gene or no gene; and
e) determining the impact of expression of the gene of interest on plant health relative to expression of the neutral control gene no gene.

126. The method of claim 125, wherein the first yeast cell and the second yeast cell is a S. cerevisiae cell.

127. The method of claim 125, wherein the gene of interest is selected from the group of a gene conferring pest resistance, herbicide resistance, stress tolerance, drought resistance, nitrogen use efficiency (NUE), disease resistance, and an ability to alter a metabolic pathway.

128. The method of claim 127, wherein the neutral control gene is selected from the group of a chloramphenicol acetyl transferase (CAT) gene, a fluorescent protein (FP) gene, a phosphomannose isomerase (PMI) gene, a β-glucuronidase (GUS) gene, a housekeeping gene, and no gene.

129. The method of claim 128, wherein the first cassette further comprises a promoter operably linked to the gene of interest for expression of the gene of interest in the first yeast cell and the second cassette further comprises a promoter operably linked to the neutral control gene or no gene for expression of the neutral control gene or no gene in the second yeast cell.

130. The method of claim 129, wherein the promoter of the first cassette and the promoter of the second cassette is the same promoter.

131. The method of claim 130, wherein determining the impact of expression of the gene of interest on plant health relative to expression of the neutral control gene or no gene is a visual observation of a yeast colony.

132. The method of claim 131, wherein the visual observation is colony size.

133. The method of claim 130, wherein determining the impact of expression of the gene of interest on plant health relative to expression of the neutral control gene or no gene is performed by hyperspectral imaging of a yeast colony.

134. The method of claim 13, wherein determining the impact of expression of the gene of interest on plant health relative to expression of the neutral control gene is performed by hyperspectral imaging of a plant tissue.

135. The method of claim 134, wherein the hyperspectral imaging of the plant tissue determines the percentage of red pixels and/or the percentage of green pixels, wherein an accumulation of red pixels indicates high levels of anthocyanin and an accumulation of green pixels indicates high levels of chlorophyll.

136. The method of claim 135, wherein the high levels of anthocyanin indicates poor plant health and the high levels of chlorophyll indicates good plant health.
